SEQUENCE LISTING (17 sequences)

SEQ ID NO: 1 - protein, 15 aa, Artificial Sequence
  synthetic peptide shown to inhibit TACE activity
TRWLVYFSRPYLVAT

SEQ ID NO: 2 - DNA, 21 bp, Artificial Sequence
  siRNA shown to inhibit TACE activity.  Also referred to as siTACE1.
ccagagacucgagaagcuutt

SEQ ID NO: 3 - DNA, 21 bp, Artificial Sequence
  siRNA shown to inhibit TACE activity.  Also referred to as siTACE2.
gcagcauucgguaagaaaatt

SEQ ID NO: 4 - DNA, 21 bp, Artificial Sequence
  siRNA shown to inhibit TACE activity.  Also referred to as siTACE3.
cgagaacaauaagauguuutt

SEQ ID NO: 5 - DNA, 23 bp, Artificial Sequence
  Primer for cloning of pro-AREG and for RT-PCR
gacctcaatgacacctactctgg

SEQ ID NO: 6 - DNA, 23 bp, Artificial Sequence
  Primer for cloning of pro-AREG and for RT-PCR
gaaatattcttgctgacatttgc

SEQ ID NO: 7 - DNA, 24 bp, Artificial Sequence
  Primer for cloning of pro-TGFalpha.
atggtcccctcggctggacagctc

SEQ ID NO: 8 - DNA, 53 bp, Artificial Sequence
  Primer for cloning of pro-TGFalpha.
tcatagatcttcttctgatataagcttttgttcgaccactgtttctgagtggc

SEQ ID NO: 9 - DNA, 22 bp, Artificial Sequence
  pBM-IRES-puro specific primer for cloning of TM mutants of AREG and TGFalpha.
tggaaaggaccttacacagtcc

SEQ ID NO: 10 - DNA, 35 bp, Artificial Sequence
  AREG deltaTM primer for cloning into pBM-IRES-puro
aaaaggatcctcattttgataaactactgtcaatc

SEQ ID NO: 11 - DNA, 34 bp, Artificial Sequence
  TGFalpha delta TM primer for cloning into pBM-IRES-puro
aaaaggatcctcaggcctgcttcttctggctggc

SEQ ID NO: 12 - DNA, 24 bp, Artificial Sequence
  RT-PCR primer for GAPDH
cccctggccaaggtcatccatgac

SEQ ID NO: 13 - DNA, 26 bp, Artificial Sequence
  RT-PCR primer for GAPDH
cataccaggaaatgagcttgacaaag

SEQ ID NO: 14 - DNA, 21 bp, Artificial Sequence
  RT-PCR primer for TACE
cagcacagctgccaagtcatt

SEQ ID NO: 15 - DNA, 23 bp, Artificial Sequence
  RT-PCR primer for TACE
ccagcatctgctaagtcacttcc

SEQ ID NO: 16 - DNA, 20 bp, Artificial Sequence
  RT-PCR primer for TGFalpha
cacactcagttctgcttcca

SEQ ID NO: 17 - DNA, 23 bp, Artificial Sequence
  RT-PCR primer for TGFalpha
tcagaccactgtttctgagtggc

(19) United States
(12) Patent Application Publication
(10) Pub. No.: US 2009/0274626 A1
(43) Pub. Date: Nov. 5, 2009

(54) TARGETING TNF-alpha CONVERTING ENZYME(TACE)- DEPENDENT GROWTH FACTOR SHEDDING IN CANCER THERAPY

(75) Inventors: Paraic A. Kenny, Templemore (IE); Mina J. Bissell, Berkeley, CA (US)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: The Regents of the University of California, Oakland, CA (US)

(21) Appl. No.: 12/022,049

(22) Filed: Jan. 29, 2008

Related U.S. Application Data
(63) Continuation of application No. PCT/US2006/030008, filed on Jul. 31, 2006.
(60) Provisional application No. 60/703,654, filed on Jul. 29, 2005.

(51) Int. Cl. A61K 49/00; A61K 31/7088; A61K 39/395; A61K 38/00; A61K 31/195; A61K 31/517; A61K 31/5377; A61K 31/506; A61K 33/24; A61K 31/337

(52) U.S. Cl. 424/9.2; 514/44 A; 424/130.1; 514/44 R; 514/2; 514/563; 514/561; 514/266.1; 514/266.4; 514/234.5; 514/266.24; 514/252.18; 424/649; 424/141.1; 514/449

(57) ABSTRACT

The invention provides methods for modulating tumor cell proliferation by contacting cells (e.g. tumor cells) with a TACE inhibitor and a compound that inhibits EGFR tyrosine kinase, whereby the TACE inhibitor enhances the sensitivity of the cell to the EGFR tyrosine kinase inhibitor. Additionally, methods for treating cancer and methods for identifying TACE inhibitors is also provided.

### CROSS-REFERENCE TO RELATED APPLICATIONS

This application claims priority to International Application No. PCT/US06/030008, filed on Jul. 31, 2006, which claimed priority to U.S. Provisional application Ser. No. 60/703,654, filed on Jul. 29, 2005, the contents of which both are incorporated herein by reference in their entirety for all purposes.

### STATEMENT OF GOVERNMENT SUPPORT

This invention was made with government support by Contract No. DE-AC02-05CH11231 awarded by the US Department of Energy, Office of Biological and Environmental Research, and an Innovator Award BC012005 and Contract No. DOD BCRP DAMD17-00-1-0224 awarded by the Department of Defense Breast Cancer Research Program. The government has certain rights in this invention.

### REFERENCE TO ATTACHED SEQUENCE LISTING

This application incorporates by reference the attached sequence listing found in paper form and in computer readable form as a .txt file, “2188_SequenceListing_ST25.txt”, both of which are hereby certified as being identical.

### BACKGROUND OF THE INVENTION

1. Field of the Invention

The present invention relates to inhibition of TNF-α Converting Enzyme (TACE) as a method for the modulation of tumor cell proliferation.

2. Related Art

The ability to proliferate independently of signals from other cell types is a fundamental characteristic of tumor cells. Whether achieved by gene overexpression, mutation, or amplification, the ability to grow independently of signals from other cell types is a central feature of tumorigenesis, and the acquisition of self-sufficiency for growth signals is a critical rate-limiting transition in the evolution of a tumor cell. Pathways downstream of the Epidermal Growth Factor Receptor (EGFR) play essential roles in cell proliferation. Genetic ablation of this receptor or of certain ligands impairs mammary gland development (Luetteke, N. C., Qiu, T. H., Fenton, S. E., Troyer, K. L., Riedel, R. F., Chang, A., and Lee, D.C. (1999). Targeted inactivation of the EGF and amphiregulin genes reveals distinct roles for EGF receptor ligands in mouse mammary gland development. Development 126, 2739-2750; Wiesen, J. F., Young, P., Werb, Z., and Cunha, G. R. (1999). Signaling through the stromal epidermal growth factor receptor is necessary for mammary ductal development. Development 126, 335-344), while deregulated ErbB pathway signaling contributes to a significant proportion of human cancer cases, both in the breast and in other tissues (Downward, J. (2003). Targeting RAS signalling pathways in cancer therapy. Nat Rev Cancer 3, 11-22). Thus, appropriate spatial and temporal regulation of EGFR signaling is crucial for correct mammary gland development and for the maintenance of mammary epithelial organization.

The HMT3522 breast cancer progression series originated from purified human breast epithelial cells derived from reduction mammoplasty. Early passages (S1) are spontaneously immortalized, non-malignant cells which require exogenous EGF for proliferation and retain the capacity to differentiate into growth-arrested, polarized acinar structures with central lumina when cultured in 3D gels of extracellular matrix (Petersen, O. W., Ronnov-Jessen, L., Howlett, A. R., and Bissell, M. J. (1992). Interaction with basement membrane serves to rapidly distinguish growth and differentiation pattern of normal and malignant human breast epithelial cells. Proc Natl Acad Sci USA 89, 9064-9068). Later passages (T4-2) grow independently of exogenous EGF and are tumorigenic in vivo (Briand, P., Nielsen, K. V., Madsen, M. W., and Petersen, O. W. (1996). Trisomy 7p and malignant transformation of human breast epithelial cells following epidermal growth factor withdrawal. Cancer Res 56, 2039-2044). They fail to arrest growth in the 3D assay and form large, continuously proliferating apolar colonies. As these cells are ERα-negative and EGFR/ERBB2-positive, they are representative of a class of breast tumors with poor prognosis (Slamon, D. J., Clark, G. M., Wong, S. G., Levin, W. J., Ullrich, A., and McGuire, W. L. (1987). Human breast cancer: correlation of relapse and survival with amplification of the HER-2/neu oncogene. Science 235, 177-182; Sommer, S., and Fuqua, S. A. (2001). Estrogen receptor and breast cancer. Semin Cancer Biol 11, 339-352). Treatment of T4-2 cells in 3D culture with inhibitors of components of the EGFR pathway elicits a striking morphological reversion of this malignant behavior and the assumption of an organized, growth-arrested, polarized acinar structure (Wang, F., Weaver, V. M., Petersen, O. W., Larabell, C. A., Dedhar, S., Briand, P., Lupu, R., and Bissell, M. J. (1998). Reciprocal interactions between beta1-integrin and epidermal growth factor receptor in three-dimensional basement membrane breast cultures: a different perspective in epithelial biology. Proc Natl Acad Sci USA 95, 14821-14826).

Tumors resulting from inappropriate activation of the EGFR are common in multiple tissues and are, for the most part, refractory to current targeted therapies. Despite the development of potent, specific EGFR inhibitors, EGFR-dependent tumors of several tissues remain a substantial clinical problem. Some patients who do respond to therapy have tumors bearing EGFR mutations (Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., et al. (2004). Activating mutations in the epidermal growth factor receptor underlying responsiveness of non-small-cell lung cancer to gefitinib. N Engl J Med 350, 2129-2139; Paez, J. G., Janne, P. A., Lee, J. C., Tracy, S., Greulich, H., Gabriel, S., Herman, P., Kaye, F. J., Lindeman, N., Boggon, T. J., et al. (2004). EGFR mutations in lung cancer: correlation with clinical response to gefitinib therapy. Science 304, 1497-1500), but this explained only a proportion of responses in these studies, and the association has not been reproduced in another large study (Tsao, M. S., Sakurada, A., Cutz, J. C., Zhu, C. Q., Kamel-Reid, S., Squire, J., Lorimer, I., Zhang, T., Liu, N., Daneshmand, M., et al. (2005). Erlotinib in lung cancer—molecular and clinical predictors of outcome. N Engl J Med 353, 133-144.). Thus, there is a need in the art to develop mechanisms to modulate tumor cell proliferation by targeting the activation of the EGFR.

### SUMMARY

The present invention provides a method for the modulation of tumor cell proliferation by the inhibition of TNF-α Converting Enzyme (TACE) and inhibition of EGFR tyrosine kinase. The present invention further provides a method for treating cancer. Additionally, the invention provides a method for identifying a TACE inhibitor.

One embodiment of the inventions provides a method for modulating cell (e.g. tumor cell) proliferation by contacting the cell with a TACE inhibitor and contacting the cell with an EGFR tyrosine kinase inhibitor, whereby the TACE inhibitor sensitizes the cells to the effect of an EGFR inhibitor. In some embodiments, the cell is in a mammal, or more specifically in a human. In some embodiments, the TACE inhibitor is an antisense nucleic acid molecule, anti-TACE antibody, siRNA oligonucleotide, soluble recombinant TACE protein fragment, small molecule, peptide, peptide mimetic or combination thereof. In some embodiments, the small molecule TACE inhibitor is (E)-2(R)-[1(S)-(Hydroxycarbamoyl)-4-phenyl-3-butenyl]-2′-isobutyl-2′-(methanesulfonyl)-4-methylvalerohydrazide (Ro 32-7315), (2R,3S)-2-([[4-(2-butynyloxy)phenyl]sulfonyl]amino)-N,3-dihydroxybutanamide (TMI-2), BMS-561392 (DPC-333), N—(R)-[2-(Hydroxyaminocarbonyl)methyl]-4-methylpentanoyl-L-naphthylalanyl-L-alanine Amide (TNF-α Protease Inhibitor-0 or TAPI-0), N—(R)-[2-(Hydroxyaminocarbonyl)methyl]-4-methylpentanoyl-L-naphthylalanyl-L-alanine, 2-aminoethyl Amide (TNF-α Protease Inhibitor-I or TAPI-1), or N—(R)-[2-(Hydroxyaminocarbonyl)methyl]-4-methylpentanoyl-L-t-butyl-alanyl-L-alanine, 2-aminoethyl Amide (TNF-α Protease Inhibitor-2 or TAPI-2). In some embodiments, the small molecule is a matrix metalloproteinase (MMP) inhibitor. In some embodiments, the method includes the further step of contacting the cell with an anilinoquinazoline compound. In some embodiments, the EGFR tyrosine kinase inhibitor is an antitumor therapeutic. Further, in some embodiments, the antitumor therapeutic is Erlotinib, Gefitinib, AG 1478, Canertinib (CI-1033), EKB-569 and Lapatinib (GW572016), Erbitux (Cetuximab), ABX-EGF, EMD-72000, Thera CIM-h-R3, or HuMax-EGFR. In some embodiments, the proliferation of the cells (e.g. tumor cells) is inhibited.

Another embodiment of the invention provides a method for treating cancer by administering to a mammal in need thereof a therapeutically effective amount of a TACE inhibitor and a therapeutically effective amount of an EGFR tyrosine kinase inhibitor, whereby the TACE inhibitor sensitizes a cell (e.g. tumor cell) to the effect of an EGFR inhibitor. In some embodiments, the EGFR tyrosine kinase inhibitor is an antitumor therapeutic. In some embodiments, the antitumor therapeutic is Erlotinib, Gleevec, Imatinib, Gefitinib, AG 1478, CEP-1347, leflunomide, Semaxanib, Imidazo[1,2-a]pyrazin-8-ylamines, Canertinib (CI-1033), EKB-569, Lapatinib (GW572016), or monoclonal antibodies that target EGFR pathway including but not limited to, Erbitux (Cetuximab), ABX-EGF, EMD-72000, Thera CIM-h-R3, HuMax-EGFR, paclitaxel, cisplatin, carboplatin, chemotherapy, and radiation treatment.

Yet another embodiment of the invention provides a method for identifying a TACE inhibitor that sensitizes a tumor cell to EGFR tyrosine kinase inhibitor by contacting a cell (e.g. tumor cell) with a compound suspected of being a TACE inhibitor, contacting the cell with an EGFR tyrosine kinase inhibitor and determining cell proliferation, whereby a compound that enhances the sensitivity of the tumor cell to the EGFR tyrosine kinase inhibitor is identified as a TACE inhibitor. In some embodiments, the cells are in a mammal. Further, in some embodiments, the mammal is a rodent. In some embodiments, the compound suspected of being a TACE inhibitor is an antisense nucleic acid molecule, anti-TACE antibody, siRNA oligonucleotide, soluble recombinant TACE protein fragment, small molecule, peptide, peptide mimetic or combination thereof. In some embodiments, the compound suspected of being a TACE inhibitor is a small molecule, which may include classes of compounds that are matrix metalloproteinase inhibitors.

### BRIEF DESCRIPTION OF THE DRAWINGS

FIG. 1. Model. A therapeutically tractable autocrine EGFR activation loop which drives the malignancy of T4-2 cells. These data suggest that TACE inhibition as an alternative strategy to kinase inhibition for tumors dependent on EGFR/ligand interaction.

FIG. 2. The EGFR Ligands. Amphiregulin and TGFα are upregulated in T4-2 cells. The relative expression levels of each member of the EGF family of ligands were screened by RT-PCR. Both Amphiregulin and TGFα were significantly upregulated in T4-2 versus S1 cells.

FIG. 3. Amphiregulin and TGFα are upregulated in T4-2 cells and can substitute for EGF to promote proliferation of S1 cells. S1 cell proliferation in the presence of each ligand is significantly different from control. A: S1 cell proliferation in the presence of equimolar EGFR ligands (860 μM) is significantly different from control. B: ELISA of CM shows that T4-2 cells secrete significantly more AREG and TGFα than S1 cells.

FIG. 4. Inhibition of sheddase activity reverts the malignant phenotype of T4-2 cells by suppressing mobilization of growth factors and downregulating EGFR pathway activity. A: Analysis of cross-sectional area of T4-2 cells treated with vehicle, AG 1478 or TAPI-2 for four days. Both AG1478 and TAPI-2 treated colonies were significantly different from controls (p<0.001). B: TAPI-2 treatment results in a dose-dependent reduction in T4-2 cell proliferation that is completely overcome by addition of soluble EGF. P-values were determined by comparison to the proliferation in 0 μM TAPI-2 in each case. C: ELISA of CM from TAPI-2-treated T4-2 cells indicating that it suppresses the shedding of both AREG and TGFα.

FIG. 5. TACE inhibitor, TAPI-2, sensitizes T4-2 cells to the effect of the EGFR inhibitor, AG1478. T4-2 cells were allowed to adhere to 48 well plates. Drugs were added at the indicated concentrations. Conditions were analyzed in triplicate. For comparison, all data were normalized to the 100%. The shift of the AG 1478 and 10 μM TAPI-2 treated cell curve to the left of the curve of the cells treated only with AG 1478 demonstrates that addition of the TACE inhibitor increases the sensitivity of the cells to the EGFR inhibitor at those doses.

FIG. 6. TACE/ADAM 17 cleaves both Amphiregulin and TGFα, and promotes T4-2 cell proliferation. A: Suppression of TACE expression reduced T4-2 cell proliferation. B: ELISA analysis of EGFR ligand shedding in T4-2 cells transfected with three siRNA oligos, either individually or as a pool. Ligand shedding is dependent on the level of TACE expression. C: ELISA analysis of the conditioned medium from the experiment showing the reversion of the malignant phenotype of T4-2 cells in 3D lrECM culture following transfection of siRNA against TACE. Levels of shed ligands are reduced by TAPI-2 treatment.

FIG. 7. TAPI-2 induced reversion of T4-2 cells is a direct result of inhibition of growth factor ectodomain shedding. A: Schematic representation of full-length and deletion mutants of Amphiregulin and TGFα. ΔTM mutants lack both the transmembrane and cytoplasmic domain and are thus secreted without requiring TACE activity. B: Analysis of cross-sectional area of T4-2 cells and derivatives in response to pharmacological inhibition of EGFR and TACE. Bar =median.

FIG. 8. Suppression of growth factor shedding by TAPI-2 in a panel of breast cancer cell lines. A: Three AREG-expressing breast cancer cell lines were identified. Equal number of cells were treated with 20 μM TAPI-2 or vehicle for 90 minutes and AREG shedding was quantified by ELISA. B: Two breast cancer cell lines expressing TGFα were identified and treated as described in (A). TAPI-2 suppresses TGFα shedding.

FIG. 9. Kaplan-Meier survival analysis of 295 human breast tumors stratified by marker expression level. High levels of (A) TACE and (B) TGFα predict poor survival. High levels of (C) Amphiregulin or (D) ERα are predictive of survival. P values represent the log-rank comparison between the upper and lower quartiles of marker expression.

### BRIEF DESCRIPTION OF THE SEQUENCES

Seq. ID No. 1. Example of peptide shown to inhibit TACE activity.

Seq. ID No. 2. Example of siRNA shown to inhibit TACE activity. Also referred to herein as siTACE1.

Seq. ID No. 3. Example of siRNA shown to inhibit TACE activity. Also referred to herein as siTACE2.

Seq. ID No. 4. Example of siRNA shown to inhibit TACE activity. Also referred to herein as siTACE3.

Seq. ID No. 5. 1^{st} Primer for cloning of pro-AREG and for RT-PCR.

Seq. ID No. 6. 2^{nd} Primer for cloning of pro-AREG and for RT-PCR.

Seq. ID No. 7. 1^{st} Primer for cloning of pro-TGFα.

Seq. ID No. 8. 2 Primer for cloning of pro-TGFα.

Seq. ID No. 9. pBM-IRES-puro specific primer for cloning of ΔTM mutants of AREG and TGFα.

Seq. ID No. 10. AREGΔTM primer for cloning into pBM-IRES-puro.

Seq. ID No. 11. TGFαΔTM primer for cloning into pBM-IRES-puro.

Seq. ID No. 12. 1^{st} RT-PCR primer for GAPDH.

Seq. ID No. 13. 2^{nd} RT-PCR primer for GAPDH.

Seq. ID No. 14. 1^{st} RT-PCR primer for TACE.

Seq. ID No. 15. 2^{nd} RT-PCR primer for TACE.

Seq. ID No. 16. 1^{st} RT-PCR primer for TGFα.

Seq. ID No. 17. 2^{nd} RT-PCR primer for TGFα.

### DETAILED DESCRIPTION OF THE INVENTION

### I. Introduction

The present invention provides a method for modulating tumor cell proliferation by contacting cells (e.g. tumor cells) with a TACE inhibitor and a compound that inhibits EGFR tyrosine kinase. Additionally, the invention provides a method for treating cancer and a method for identifying TACE inhibitors. The invention is based on the surprising discovery that inhibition of protease, TACE/ADAM 17, sensitizes tumor cells to an EGFR tyrosine kinase inhibitor, i.e. reverting the malignant phenotype by preventing mobilization of two crucial growth factors, Amphiregulin and TGFa. It is also based on the discovery that the efficacy of EGFR inhibitors is overcome by physiological levels of growth factors and that successful EGFR inhibition is dependent on reducing ligand bioavailability, which can be achieved by inhibition of TACE.

### II. Definitions

The term “TACE” refers to Tumor Necrosis Factor a Converting Enzyme, which is also known as ADAM17. The mRNA and amino acid sequences for Homo sapiens can be found under GenBank accession number NM_{—}003183. The gene encodes an 824-amino acid polypeptide containing the features of the ADAM family: a secretory signal sequence, a disintegrin domain, and a metalloprotease domain. Expression studies showed that the encoded protein cleaves precursor tumor necrosis factor-alpha to its mature form.

The term “EGFR” refers to Epidermal Growth Factor Receptor which is a tyrosine protein kinase. The EGFR molecule has 3 regions: one projects outside the cell and contains the site for binding EGF; the second is embedded in the membrane; the third projects into the cytoplasm of the cell's interior. EGFR is a kinase that attaches phosphate groups to tyrosine residues in proteins. The mRNA and amino acid sequences for Homo sapiens can be found under GenBank accession numbers NM-005228, NM_{—}201282, NM_{—}201283, or NM_{—}201284.

The term “EGFR ligand” refers to a molecule which binds and activates EGFR tyrosine kinase. By activate, it is intent to mean that the EGFR tyrosine kinase activity is 5%, 10%, 15%, 20%, 25%, 30%, 35%, 40%, 45%, 50%, 55%, 60%, 65%, 70%, 75%, 80%, 85%, 90%, 95%, or 98% higher relative to activity in the absence of the EGFR ligand. Examples of EGFR ligands include HB-EGF, Epiregulin, Amphiregulin, EGF, Neuregulin 1, Cripto, Neuregulin 2, or TGFα.

The term “TACE inhibitor” refers to a compound that inhibits TACE activity. By inhibit, it is intended to mean that the TACE activity is 5%, 10%, 15%, 20%, 25%, 30%, 35%, 40%, 45%, 50%, 55%, 60%, 65%, 70%, 75%, 80%, 85%, 90%, 95%, 96%, 97% or 98% less activity relative to activity in the absence of the inhibitor. Such a TACE inhibitor may be an antisense nucleic acid molecule, anti-TACE antibody, siRNA, soluble recombinant TACE protein fragment, small molecule, peptide, or peptide mimetic.

The term “antitumor therapeutic” means compound that inhibits EGFR tyrosine kinase. Examples of such an antitumor therapeutic are Erlotinib, Gleevec, Imatinib, Gefitinib, AG 1478, CEP-1347, leflunomide, Semaxanib, Imidazo[1,2-a]pyrazin-8-ylamines, Canertinib (CI-1033), EKB-569, Lapatinib (GW572016), or monoclonal antibodies that target EGFR pathway including but not limited to, Erbitux (Cetuximab), ABX-EGF, EMD-72000, Thera CIM-h-R3, HuMax-EGFR, paclitaxel, cisplatin, carboplatin, chemotherapy, and radiation treatment.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence.

The term “homology” or “homologous” means an amino acid similarity measured by the program, BLAST (Altschul et al (1997), “Gapped BLAST and PSI-BLAST: a new generation of protein database search programs”, Nucleic Acids Res. 25:3389-3402), as found at http://www.ncbi.nlm.nih.gov/blast/Blast.cgi and expressed as—(% identity n/n). In measuring homology between a peptide and a protein of greater size, homology is measured only in the corresponding region; that is, the protein is regarded as only having the same general length as the peptide, allowing for gaps and insertions.

The term “effective amount” herein refers to an amount sufficient to elicit the desired biological response (e.g. modulation of cell proliferation).

The term “therapeutically effective amount” means the amount required to modulate (e.g. inhibit) the proliferation, development, growth or metastasis of cancerous cells; reduction of tumor size and growth rate, prolonged survival rate, reduction in concurrent cancer therapeutics administered to patient. By modulate, it is intended to mean tumor cell proliferation, development, growth or metastasis of cancerous cells is altered 25%, 30%, 35%, 40%, 45%, 50%, 55%, 60%, 65%, 70%, 75%, 80%, 85%, 90%, 95%, 96%, 97% or 98% relative to the level of tumor cell proliferation, development, growth or metastasis of cancerous cells in the absence of a compound.

The term “subject” herein refers to any vertebrate species. Particularly preferred subjects are mammals, with humans being the most preferred subject.

The term “conservative substitution” means a substitution where an amino acid residue is substituted for another in the same class, where the amino acids are divided into non-polar, acidic, basic and neutral classes, as follows: non-polar:(A), Val (V), Leu (L), Ile (I), Phe (F), Trp (W), Pro (P), Met (M); acidic: Asp (D), Glu (E); basic: Lys (K), Arg (R), H is (H); uncharged polar: Gly (G), Ser (S), Thr (T), Cys (C), Asn (N), Gln (Q), Tyr (Y). A non-conservative amino acid substitution is one where the residues do not fall into the same class, for example, substitution of a basic amino acid for a neutral or non-polar amino acid.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The term “antibody” refers to a polypeptide encoded by an immunoglobulin gene or functional fragments thereof that specifically binds and recognizes an antigen (e.g., TACE or EGFR). The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′.sub.2, a dimer of Fab which itself is a light chain joined to V.sub.H-C.sub.H1 by a disulfide bond. The F(ab)′.sub.2 may be reduced under mild conditions to break the disulfide linkage in the hinge region, thereby converting the F(ab)′.sub.2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

### III. Method of Modulating Cell Proliferation

One embodiment of the invention provides methods for modulating (e.g. inhibiting or enhancing) proliferation of cells (e.g. tumor cells). The method comprises contacting cells (e.g. tumor cells) expressing TACE with a TACE inhibitor and contacting the cells with a compound that inhibits EGFR tyrosine kinase, whereby the TACE inhibitor enhances the sensitivity of cells (e.g. tumor cells) to the EGFR inhibitor.

By inhibit, it is intended to mean that TACE activity or EGFR tyrosine kinase activity is 5%, 10%, 15%, 20%, 25%, 30%, 35%, 40%, 45%, 50%, 55%, 60%, 65%, 70%, 75%, 80%, 85%, 90%, 95%, 96%, 97%, 98% less with the compound than activity relative to activity in the absence of the compound.

By sensitivity, it is intended to mean that there is an increased susceptibility of cells to modulation (e.g. inhibition) of the proliferation, development, growth or metastasis of cancerous cells; reduction of tumor size and growth rate, prolonged survival rate, or reduction in concurrent cancer therapeutics administered to patient.

By enhanced, it is intended to mean that lower concentrations of EGFR inhibitor would be required when administered with the TACE inhibitor to achieve the same effect with EGFR inhibitor alone. Sensitivity of the cell to the compound that inhibits EGFR tyrosine kinase is enhanced (i.e. reduced concentrations required needed to achieve the same effect) when the EGFR inhibitor has 5%, 10%, 15%, 20%, 25%, 30%, 35%, 40%, 45%, 50%, 55%, 60%, 65%, 70%, 75%, 80%, 85%, 90%, 95%, 96%, 97%, 98% greater effectiveness (i.e. modulation of the proliferation, development, growth or metastasis of cancerous cells; reduction of tumor size and growth rate, prolonged survival rate, reduction in concurrent cancer therapeutics administered to patient is reduced) relative to the sensitivity of the cell in absence of the TACE inhibitor.

The TACE inhibitor and EGFR inhibitor may be administered simultaneously or sequentially. For example, the TACE inhibitor may be administered first, followed by the EGFR inhibitor. Alternatively, the EGFR inhibitor may be administered first, followed by the TACE inhibitor. In some cases, the TACE inhibitor and the EGFR inhibitor are administered in the same formulation. In other cases the TACE inhibitor and the EGFR inhibitor are administered in different formulations. When the TACE inhibitor and the EGFR inhibitor are administered in different formulations, their administration may be simultaneous or sequential.

In some embodiments, TACE inhibitor is delivered sequentially or in combination with known anilinoquinazoline compounds (e.g. AG1478, Erlotinib or Gefitinib) and EGFR inhibitors, thereby improving the efficacy of these compounds and inhibitors in rate of inhibition of EGFR, Erb2 and other related proteins to inhibit tumor progression and growth.

a. TACE Inhibition

In one embodiment, known methods are used to identify compounds that inhibit TACE. Such inhibitors include antisense nucleic acid molecules, anti-TACE antibodies, siRNA oligonucleotides, soluble recombinant TACE protein fragments, small molecules, peptides, peptide mimetics or any combination thereof.

1. Small Molecules

In one embodiment, the TACE inhibitors are small molecules. Examples of specific and orally active TACE inhibitors include those developed by Roche (Beck, G., Bottomley, G., Bradshaw, D., Brewster, M., Broadhurst, M., Devos, R., Hill, C., Johnson, W., Kim, H. J., Kirtland, S., et al. (2002). (E)-2(R)-[1(S)-(Hydroxycarbamoyl)-4-phenyl-3-butenyl]-2′-isobutyl-2′-(methanesulfonyl)-4-methylvalerohydrazide (Ro 32-7315), a selective and orally active inhibitor of tumor necrosis factor-alpha convertase. J Pharmacol Exp Ther 302, 390-396) and Wyeth (Zhang, Y., Hegen, M., Xu, J., Keith, J. C., Jr., Jin, G., Du, X., Cummons, T., Sheppard, B. J., Sun, L., Zhu, Y., et al. (2004). Characterization of (2R,3S)-2-({[4-(2-butynyloxy)phenyl]sulfonyl}amino)-N,3-dihydroxybutanamide, a potent and selective inhibitor of TNF-alpha converting enzyme. Int Immunopharmacol 4, 1845-1857) for the treatment of arthritis, (E)-2(R)-[1(S)-(Hydroxycarbamoyl)-4-phenyl-3-butenyl]-2′-isobutyl-2′-(methanesulfonyl)-4-methylvalerohydrazide (Ro 32-7315) and (2R,3S)-2-([[4-(2-butynyloxy)phenyl]sulfonyl]amino)-N,3-dihydroxybutanamide (TMI-2), respectively. Another example of a specific TACE inhibitor is that Bristol Myers-Squibb's tumor necrosis factor-alpha (TNF alpha) converting enzyme inhibitor BMS-561392 (DPC-333) for the potential treatment of diseases characterized by overproduction of TNF alpha, such as rheumatoid arthritis (RA) (Grootveld M, McDermott M F., Curr Opin Investig Drugs. 2003 May; 4(5):598-602). Other examples of specific TACE inhibitors are N—(R)-[2-(Hydroxyaminocarbonyl)methyl]-4-methylpentanoyl-L-naphthylalanyl-L-alanine Amide (TNF-α Protease Inhibitor-0 or TAPI-0), N—(R)-[2-(Hydroxyaminocarbonyl)methyl]-4-methylpentanoyl-L-naphthylalanyl-L-alanine, 2-Page 13 of 55 aminoethyl Amide (TNF-α Protease Inhibitor-I or TAPI-1), or N—(R)-[2-(Hydroxyaminocarbonyl)methyl]-4-methylpentanoyl-L-t-butyl-alanyl-L-alanine, 2-aminoethyl Amide (TNF-α Protease Inhibitor-2 or TAPI-2).

In one embodiment, the TACE inhibitor compound is a matrix metalloproteinase inhibitor. Examples of compounds which may TACE inhibitors are compounds that inhibit matrix metalloproteinases include but are not limited to GM6001, batimastat, marimastat, CT11746, KB-R7785, prinomastat, BAY129566, Ro32-3,555 and CGS27023A. In some embodiments, the TACE inhibitor compound is an ADAM inhibitor.

The present invention also incorporates by reference those compounds known to be TACE inhibitors including but not limited to hydantoin, hydantoin derivatives or related heterocycles (see e.g. U.S. Pat. No. 7,041,693, U.S. Pat. No. 6,906,053, U.S. Pat. No. 6,890,915, U.S. Patent Pub. No. 20060063818, U.S. Patent Pub. No. 20050171096, U.S. Patent Pub. No. 20040254231, U.S. Patent Pub. No. 20040209874, U.S. Patent Pub. No. 20040067996 and U.S. Patent Pub. No. 20030130273); cyclic sulfone derivatives (see e.g. U.S. Pat. No. 7,015,217 and U.S. Patent Pub. No. 20030149031); spiro-cyclic 1-amino acid derivatives (see e.g. U.S. Pat. No. 6,962,938, U.S. Pat. No. 6,720,329, U.S. Patent Pub. No. 20040132693 and U.S. Patent Pub. No. 20030087882); hydroxamates and hydroxamic acid derivatives (see e.g. U.S. Pat. No. 7,034,057, U.S. Pat. No. 6,946,473, U.S. Pat. No. 6,825,354, U.S. Pat. No. 6,812,227, U.S. Pat. No. 6,770,647, U.S. Pat. No. 6,762,178, U.S. Pat. No. 6,740,649, U.S. Pat. No. 6,716,833, U.S. Pat. No. 6,548,524, U.S. Pat. No. 6,534,491, U.S. Pat. No. 6,498,167, U.S. Pat. No. 6,340,691, U.S. Pat. No. 6,326,516, U.S. Pat. No. 6,277,885, U.S. Pat. No. 6,225,311, U.S. Pat. No. 6,200,996, U.S. Pat. No. 6,197,795, U.S. Pat. No. 6,162,821, U.S. Pat. No. 6,162,814, U.S. Pat. No. 5,977,408, U.S. Pat. No. 5,962,481, U.S. Pat. No. 5,929,097, U.S. Patent Pub. No. 20050215549, U.S. Patent Pub. No. 20050113346, U.S. Patent Pub. No. 20040127524, U.S. Patent Pub. No. 20040033988, U.S. Patent Pub. No. 20030212049, U.S. Patent Pub. No. 20030208066, U.S. Patent Pub. No. 20030181441, U.S. Patent Pub. No. 20030139388, U.S. Patent Pub. No. 20030130257, U.S. Patent Pub. No. 20030130238, U.S. Patent Pub. No. 20030008849, U.S. Patent Pub. No. 20020188132, U.S. Patent Pub. No. 20020147342, U.S. Patent Pub. No. 20020132826, U.S. Patent Pub. No. 20010056088, U.S. Patent Pub. No. 20010051614, U.S. Patent Pub. No. 20010046989 and U.S. Patent Pub. No. 20010025047); Barbituric acid derivatives (see e.g. U.S. Pat. No. 6,936,620, U.S. Patent Pub. No. 20030229084 and U.S. Patent Pub. No. 20030166647); bicyclic lactam derivatives (see e.g. U.S. Pat. No. 6,884,806 and U.S. Patent Pub. No. 20030181438); Acetylenic sulfonamide thiol tace and derivatives (see e.g. U.S. Pat. No. 6,313,123 and U.S. Patent Pub. No. 20020103163); N-sulfonylpiperidines (see e.g. U.S. Patent Pub. No. 20060142336); sulphonamide derivatives (see e.g. U.S. Patent Pub. No. 20050256176); substituted 1,3-dihydro-imidazol-2-one and its derivates (see e.g. U.S. Patent Pub. No. 20050075384); uracil derivatives (see e.g. U.S. Patent Pub. No. 20030229081); beta-sulfone derivatives (see e.g. U.S. Patent Pub. No. 20030212056); or other small molecules (see e.g. U.S. Patent Pub. No. 20040186088).

Thus, in a preferred embodiment, TACE inhibitor compounds are small molecules, including specific and orally active TACE inhibitors and their derivatives and analogs, are delivered to a subject for TACE inhibition to reduce of the levels of circulating growth factors by inhibiting growth factor shedding to prevent tumor growth and progression. In a preferred embodiment, it may be preferred that the manufacturers' directions as to administration and delivery be followed. However, it is anticipated that lower dosages and different delivery routes and methods may be used in accordance to dosages and delivery used for neoplastic therapies and treatments as is known in the art.

2. Peptides

In another embodiment, the TACE inhibitors are peptides. For example the peptide, TRWLVYFSRPYLVAT, has been shown to bind to TACE and inhibit the TNF-alpha release from LPS-stimulated human peripheral blood mononuclear cells (PBMC) (Lovering F, Zhang Y, Therapeutic potential of TACE inhibitors in stroke, Curr Drug Targets CNS Neurol Disord. 2005 April; 4(2):161-8), can be used to inhibit TACE. In one embodiment, the peptide, TRWLVYFSRPYLVAT, or a sequence having a percent identity of at least 70%, 75%, 80%, 85%, more preferably 90%, 95%, and even more preferably 97%, 98%, 99%, 100%, is delivered to a subject to reduce of the levels of circulating growth factors by inhibiting growth factor shedding to prevent tumor growth and progression. In another embodiment, the invention also encompasses nucleic acids which encode the inhibitory TACE peptides.

The peptides may be made and purified by methods known in the art, preferably by in vitro automated synthesis, but also by recombinant DNA methods. Furthermore, these peptides can be synthesized using L-amino acids, non-natural or other modified amino acids, as is known in the art, in order to synthesize peptides which can act upon targets in the body and be degraded, yet do not interfere with normal protein function. The peptides can be stored in lypholized form and dissolved in aqueous buffers or water prior to use. For the purposes of experimental use, the peptides are dissolved in sterilized degassed buffers to optimize biological activity which remains stable over 1-6 months at 4° C.

The TACE inhibitory peptides may be prepared according to known pharmaceutical technology. They may be administered singly or in combination, and may further be administered in combination with other cardiovascular drugs as known and determined by those familiar with the art. They may be conventionally prepared with excipients and stabilizers in sterilized, lyophilized powdered form for injection, or prepared with stabilizers and peptidase inhibitors of oral and gastrointestinal metabolism for oral administration. It is further contemplated that the peptides of the invention can be administered by methods including, but not limited to, intravenous, infusion, rectal, inhalation, transmucosal or intramuscular administration.

3. siRNA Oligonucleotides

In another embodiment, siRNA is used to inhibit TACE. siRNAs were generated against the following TACE sense strand sequences: CCAGAGACUCGAGAAGCUUtt (siTACE1; SEQ ID NO:2), GCAGCAUUCGGUAAGAAAAtt (siTACE2; SEQ ID NO:3) and CGAGAACAAUAAGAUGUUUtt (siTACE3; SEQ ID NO:4). In one embodiment, each individual siRNA can be transfected either individually or in combination at about 10 nM to about 250 nM. In a preferred embodiment, each individual siRNA can be transfected either individually or in combination at about 50 nM to about 200 nM, and even more preferably at about 75 nM to about 150 nM. In another embodiment, any combination of the three siRNAs can be transfected wherein the total of the siRNAs is about 10 nM to 250 nM. In a preferred embodiment, any combination of the three siRNAs can be transfected wherein the total of the siRNAs is about 50 nM to about 200 nM, and even more preferably at about 75 nM to about 150 nM.

siRNA can be made using methods and algorithms such as those described by Wang L, Mu F Y. (2004) A Web-based Design Center for Vector-based siRNA and siRNA cassette. Bioinformatics. 20(11): 1818-20; Khvorova A, Reynolds A, Jayasena S D. (2003) Functional siRNAs and miRNAs exhibit strand bias. Cell. 115(2):209-16; Harborth J, Elbashir S M, Vandenburgh K, Manning a H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat. Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (http://www.idtdna.com/SciTools/SciTools.aspx), or siDESIGN™ Center from Dharmacon, Inc. (http://design.dharmacon.com/default.aspx?source=0). siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

4. Antisense Oligonucleotides

In another embodiment, antisense oligonucleotides (“oligos”) can be designed to inhibit TACE activity. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the Rac1b and other candidate gene antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

5. Antibodies

In other embodiments, such antibodies that specifically bind or inhibit TACE, may be used to inhibit TACE. Such use of antibodies has been demonstrated by others and may be useful in the present invention to inhibit or downregulate TACE.

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

b. EGFR Tyrosine Kinase Inhibitors

TACE inhibitor is administered in conjunction with an agent that inhibits EGFR tyrosine kinase activity for treating or preventing EGFR-dependent cancers. Such agents include small molecules, antisense nucleic acids, siRNA oligonucleotides, anti-EGFR antibodies, peptides and peptide mimetics. Such small molecule inhibitors include but are not limited to, Erlotinib, Gefitinib, AG 1478, Canertinib (CI-1033), EKB-569 and Lapatinib (GW572016). Monoclonal antibodies that target EGFR pathway include but are not limited to, Erbitux (Cetuximab), ABX-EGF, EMD-72000, Thera CIM-h-R3, and HuMax-EGFR.

c. Tumor Cells

The present method can be used on tumors resulting from inappropriate activation of the EGFR pathway. EGFR-dependent tumors are common in multiple tissues. Tissues which typically have EGFR-dependent tumors include breast tissue, lung tissue, colon tissue or pancreatic tissue. Examples of other tissues which can have EGFR-dependent tumors include but are not limited to skin, kidney, brain, liver, thyroid and prostate. This is not intended to limit the scope of types of tissue cells, rather those with knowledge and skill in the art will appreciate other cells on which the present method may be utilized.

The method can be used on any cell that expresses TACE. Preferably, the cell is in a mammal. Examples of mammals include but are not limited to rodents, rabbits, primates, humans, dogs, cats, or farm animals (e.g. horses, cows, sheep, pigs, etc.).

### IV. Method of Treating Cancer

Another embodiment of the invention provides a method for the treatment of cancer of a mammal in need of a therapeutically effective amount of a TACE inhibitor and a therapeutically effective amount of an EGFR tyrosine kinase inhibitor, whereby the TACE inhibitor enhances the sensitivity of the cell to the EGFR inhibitor.

The TACE inhibitor and EGFR inhibitor may be administered simultaneously or sequentially. For example, the TACE inhibitor may be administered first, followed by the EGFR inhibitor. Alternatively, the EGFR inhibitor may be administered first, followed by the TACE inhibitor. In some cases, the TACE inhibitor and the EGFR inhibitor are administered in the same formulation. In other cases the TACE inhibitor and the EGFR inhibitor are administered in different formulations. When the TACE inhibitor and the EGFR inhibitor are administered in different formulations, their administration may be simultaneous or sequential.

TACE inhibitor is administered in conjunction with a therapeutic agent that inhibits EGFR tyrosine kinase activity for treating or preventing EGFR-dependent cancers. Such agents include small molecules, antisense nucleic acids, siRNA oligonucleotides, anti-EGFR antibodies, peptides and peptide mimetics. Such small molecule inhibitors include but are not limited to, Erlotinib, Gefitinib, AG 1478, Canertinib (CI-1033), EKB-569 and Lapatinib (GW572016). Monoclonal antibodies that target EGFR pathway include but are not limited to, Erbitux (Cetuximab), ABX-EGF, EMD-72000, Thera CIM-h-R3, and HuMax-EGFR.

In some embodiments, the TACE inhibitor is an antisense nucleic acid molecule, anti-TACE antibodie, siRNA, soluble recombinant TACE protein fragment, small molecule, peptide, peptide mimetic or combination thereof.

In some embodiments, the EGFR inhibitor is an antitumor therapeutic. The antitumor therapeutic is Erlotinib, Gleevec, Imatinib, Gefitinib, AG 1478, CEP-1347, leflunomide, Semaxanib, Imidazo[1,2-a]pyrazin-8-ylamines, Canertinib (CI-1033), EKB-569, Lapatinib (GW572016), or monoclonal antibodies that target EGFR pathway including but not limited to, Erbitux (Cetuximab), ABX-EGF, EMD-72000, Thera CIM-h-R3, HuMax-EGFR, paclitaxel, cisplatin, carboplatin, chemotherapy, and radiation treatment.

In one embodiment, the method is used to treat cancers caused by tumors resulting from inappropriate activation of the EGFR pathway which is common in multiple tissues. Breast cancer, lung cancer, colon cancer and pancreatic cancer are examples of cancers caused by the inappropriate activation of EGFR. Other cancers caused by the activation of EGFR (e.g skin, kidney, brain, liver, thyroid and prostate) can also be treated with the present method.

In one embodiment, the method may be used to treat a mammal with a cancer caused by tumors resulting from inappropriate activation of the EGFR pathway. Examples of mammals include rodents, rabbits, primates, humans, dogs, cats, or farm animals (e.g. horses, cows, sheep, pigs, etc.).

In a further embodiment, a TACE inhibitor is delivered sequentially or in combination with known anilinoquinazoline compounds and EGFR inhibitors, thereby improving the efficacy of these compounds and inhibitors in rate of inhibition of EGFR, Erb2 and other related proteins to inhibit tumor progression and growth.

In one embodiment, these compounds may prove efficacious in selected subsets of patients, one such cohort being those who depend on TACE-dependent autocrine stimulation of EGFR/ErbB2.

a. Routes of Administration/Formulations

The TACE inhibitor and EGFR tyrosine kinase inhibitor can be used to treat or prevent a variety of disorders associated with cancer. The small molecules, antibodies, peptides and nucleic acids are administered to a patient in an amount sufficient to elicit a therapeutic response in the patient (e.g., inhibiting the development, growth or metastasis of cancerous cells; reduction of tumor size and growth rate, prolonged survival rate, reduction in concurrent cancer therapeutics administered to patient). An amount adequate to accomplish this is defined as “therapeutically effective dose or amount.”

The small molecules, antibodies, peptides and nucleic acids of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the small molecules, antibodies, peptides and nucleic acids of the invention can be in any convenient manner, e.g., by injection, intratumoral injection, intravenous and arterial stents (including eluting stents), cather, oral administration, inhalation, transdermal application, or rectal administration. In some cases, the small molecules, antibodies, peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington 's Pharmaceutical Sciences, 17^{th} ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector (e.g., small molecule, antibody, peptide, or nucleic acid) to be administered in the treatment or prophylaxis of diseases or disorder associated with the disease, the physician evaluates circulating plasma levels of the small molecule, antibody, polypeptide or nucleic acid, polypeptide or nucleic acid toxicities, progression of the disease (e.g., cancer). The dose for a small molecule is generally from about 0.1 to about 100 mg per kg, preferably from about 0.2 to about 50 mg per kg, most preferably from about 0.5 to about 25 mg per kg body weight. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked nucleic acid is from about 1 μg to about 100 μg for a typical 70 kilogram patient, and doses of vectors which include a viral particle are calculated to yield an equivalent amount of therapeutic nucleic acid.

In another embodiment, dosage levels are of the order of from about 0.1 milligram to about 140 milligram per kilogram of body weight per day are useful in treatment (about 0.5 milligram to about 7 gram per human patient per day). The amount of active ingredient that may be combined with the carrier materials to produce a single dosage form will vary depending upon the host treated and the particular mode of administration. Dosage unit forms will generally contain between from about 1 milligram to about 500 milligram of an active ingredient.

For administration, small molecules, antibodies, polypeptides and nucleic acids of the present invention can be administered at a rate determined by the LD-50 of the small molecule, antibody, polypeptide or nucleic acid, and the side-effects of the small molecule, antibody, polypeptide or nucleic acid at various concentrations, as applied to the mass and overall health of the patient. Administration can be accomplished via single or divided doses, e.g., doses administered on a regular basis (e.g., daily, weekly, monthly) for a period of time (e.g., 2, 3, 4, 5, 6, 7 days or 1-3 weeks or 1-3 months or more).

In certain circumstances it will be desirable to deliver the pharmaceutical compositions comprising the TACE inhibitor of the present invention parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators, including Dharmacon, but typically require complex formulation knowledge. There are a few reports that cite success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

1. Liposomes/Nanocapsules

In certain embodiments, the inventors contemplate the use of liposomes, nanocapsules, microparticles, microspheres, lipid particles, vesicles, and the like, for the administration of the TACE inhibitors of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al, 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 A, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684).

2. Gene Therapy

In certain embodiments, the nucleic acids encoding inhibitory TACE peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory TACE peptides and nucleic acids of the present invention, thereby mitigating the effects of over amplification of a candidate gene associated with reduced survival rate.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11: 167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith(1994) Gene Therapy 1:165-169; and Zhou et al. (1994) J. Exp. Med. 179:1867-1875). Additional suitable vectors include E1B gene-attenuated replicating adenoviruses described in, e.g., Kim et al., Cancer Gene Ther. 9(9):725-36 (2002) and nonreplicating adenovirus vectors described in e.g., Pascual et al., J. Immunol. 160(9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory TACE nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9(8):692-9 (2002).

### V. Method of Identifying Compound that Inhibits Tumor Cell Proliferation

Another embodiment of the invention provides a method for identifying a TACE inhibitor that sensitizes a tumor cell to EGFR tyrosine kinase inhibitor. The method comprises contacting a cell (e.g. tumor cell) with a compound suspected to be a TACE inhibitor and an EGFR tyrosine kinase inhibitor, and determining cell proliferation. If a compound suspected of being a TACE inhibitor enhances the sensitivity of the tumor cell to the EGFR tyrosine kinase inhibitor, that compound is identified as a TACE inhibitor.

Cell proliferation is determined by methods appreciated to those skilled in the art. Examples of methods to determine cell proliferation include but are not limited to: clonogenic assays in which cells are plated at low densities and the number of colonies is scored after a growth period; permeability assays which involve staining damaged cells with dye and counting, either manually using a hemocytometer or mechanically using a flow cytometer, viable cells that exclude the dye; metabolic activity assays which measure by spectrophotometry dyes converted by cells uptaking tetrazolium salts; and direct proliferation assays using DNA synthesis as an indicator of cell growth. Types of commercially-available, specific cell proliferation assay kits are also available and are hereby incorporated by reference. Examples of these kits include but are not limited to MTT Cell Proliferation Assay (ATCC Bioproducts™), CellTiter 96® AQueous One Solution Cell Proliferation Assay (Promega Corporation), Cell Proliferation Kit 1—MTT (Roche Applied Science), Cell Proliferation Kit II-XTT (Roche Applied Science), 5′-Bromo-2′-deoxy-uridine Labeling and Detection Kit III (Roche Applied Science), Cell Proliferation ELISA, BrdU—colorimetric/chemiluminescence (Roche Applied Science), HitKit® HCS Reagent Kit (Cellomics), Cell Growth Determination Kit—MTT based (Sigma-Aldrich), and Cell Proliferation Assay Kit—WST dye/ELISA based (Chemicon International).

In some embodiments, compounds suspected to be TACE inhibitors are antisense nucleic acid molecules, anti-TACE antibodies, siRNA oligonucleotides, soluble recombinant TACE protein fragments, small molecules, peptides and peptide mimetics. In some embodiments, the compound suspected of being a TACE inhibitor is a matrix metalloproteinase (MMP) inhibitor or an ADAM inhibitor.

In a preferred embodiment, the EGFR inhibitor is AG 1478.

In some embodiments, the cell (e.g. tumor cell) is in a mammal. The mammal may be a rodent, primate, human, dog, cat, or farm animal (e.g. horse, cow, sheep, pig, etc.).

Examples of ways in which the compound can be identified are through high throughput screening, a TACE FRET assay or by an in vitro TACE assay. Those with knowledge and skill in the art will appreciate other methods of screening compounds.

a. High Throughput Screening

In one embodiment, high throughput screening (HTS) methods are used to identify compounds that inhibit tumor cell proliferation, e.g. identifying compounds that inhibit TACE. HTS methods involve providing a combinatorial chemical or peptide library containing a large number of potential therapeutic compounds. Such potential therapeutic compounds would include but are not limited to compounds known to inhibit TACE, classes of compounds known to inhibit TACE and compounds believed to inhibit TACE. Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., ECIS™, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

b. TACE FRET Assay

TACE inhibitor compounds can be screened based on their ability to inhibit the cleavage of the substrate by the purified enzyme in a fluorescence-based FRET assay as described in Zhang et al., J. Pharmacol. Exp. Ther. 309(1):348-355 (2004), Jin et al, Anal. Biochem. 302(2):268-275 (2002), and Zhang et al., Int. Immunopharmacol. 4:1845-1857 (2004), all of which are hereby incorporated by reference.

In this assay, the catalytic domain of TACE is pretreated with compounds suspected of being TACE inhibitors for 10 minutes at room temperature. The reaction is initiated by the addition of pro-TNF-α peptide to the TACE protein. Any increase or decrease in fluorescence is monitored at excitation of 320 nm and emission of 420 nm over a period of 10 minutes.

c. In Vitro TACE Assay

TACE inhibitor compounds can be screened by measuring TACE activity in the presence of a suspected inhibitor based on the in vitro TACE assay described in Beck et al., J. Pharmacol. Exp. Ther. 302(1):390-396 (2002), which is hereby incorporated by reference.

In this assay, a recombinant form of TACE, which lacks the transmembrane region and cytoplasmic tail is used. Compounds suspected of being TACE inhibitors are added to the recombinant TACE. TACE activity is partially purified from concentrated culture media by Q-Sepharose®, concanavali A Sepharose®, and Superdex™ 75 chromatography. TACE activity is determined by measuring the production of the peptide product from a peptide substrated with Dpa, where Dpa is N-3-(2,4-di-nitrophenyl)-L-2,3-diaminopropionyl. The assay is carried out in 10 mM Tris-HCL (pH 8.0), 50 mM NaCl, and 2% octylglycoside and at a substrated concentration of 100 μM. After 60 minutes at 37° C., the reaction is stopped by adding acetic acid to a final concentration of 1M. The peptide product is separated form the reaction mixture by reverse-phase high performance liquid chromatography, using a 28-70% acetonitrile gradient on a C₈ column. The absorbance of the eluate at 360 nm is measured as an index of the amount of product formed.

### VI. Examples

The following examples are meant to exemplify and illustrate, but not to limit the invention.

### Example 1

### Materials and Methods

Cell culture All reagents were purchased from SIGMA (St. Louis, Mo.) except where otherwise noted. HMT3522 cells were cultivated on 2D and 3D substrata in H14 medium, a 50:50 mix of DMEM/F12 (UCSF Cell culture Facility, San Francisco, Calif.) supplemented with 5 μg/ml prolactin, 250 ng/ml insulin, 1.4×10⁻⁶ M hydrocortisone, 10⁻¹⁰ M β-estradiol, 2.6 ng/ml sodium selenite and 10 μg/ml transferrin. S1 cells were additionally supplemented with 10 ng/ml EGF. In various experiments, cells were supplemented with AREG, TACE, TGFα. In all cases, AREG and TGFα were used at the same molar concentration as EGF (860 μM).

For 3D lrECM culture, T4-2 cells were seeded at 21000 cells per cm² on top of Matrigel, overlaid with H14 medium containing 5% Matrigel (BD Biosciences, San Jose, Calif.), and treated with 80 nM AG1478, 20 μM TAPI-2 or the relevant vehicle controls.

Amphotropic retroviruses were generated by transfection (Lipofectamine; Invitrogen, Carlsbad, Calif.) of the Phoenix packaging cell line (a gift of Dr. Gary Nolan, Stanford) with pBM-IRES-Puro or derivatives containing the AREG or TGFα open reading frames. Two million phoenix cells per 6 cm dish were plated the day prior to transfection and transfected with 2 μg of the appropriate retroviral construct. Retrovirus-containing culture medium was harvested after 48 hrs, supplemented with polybrene to 5 μg/ml and added to HMT3522 cells at 30-50% confluence. Pools of stable infectants were selected in 1 μg/ml puromycin.

Silencer™ siRNAs against TACE (Ambion, Austin, Tex.) were co-transfected with pEGFP-C1 (BD Biosciences). T4-2 cells were trypsinized post-transfection and plated at low density. Proliferation was assessed by counting the transfected (green) cells per colony after four days. Random siRNA sequence was used as a negative control.

Silencer™ siRNAs against TACE (Ambion, Austin, Tex.) were also transfected in according to the Reverse Transfection protocol of Invitrogen. siRNAs against the following TACE sense strand sequences were transfected either individually (100 nM) or as a pool (33.3 nM each): CCAGAGACUCGAGAAGCUUtt (SEQ ID NO: 2), GCAGCAtUCGGUAAGAAAAtt (SEQ ID NO:3) and CGAGAACAAUAAGAUGTUUtt (SEQ ID NO: 4). For 3D culture assays, T4-2 cells were trypsinized post-transfection and plated as single cells in Matrigel. Knockdown was assessed by western blot from a parallel transfection 48 hrs post-transfection. Random siRNA sequence was used as a negative control.

Indirect immunofluorescence. Colonies were solubilized from MATRIGEL culture by shaking in PBS/0.05M EDTA on ice for 30 mins, fixed in 4% paraformaldehyde, permeabilized, stained with anti-α6-integrin (Chemicon, Temecula, Calif.) and counterstained with DAPI.

Western blotting. Cells were lysed in 50 mM Tris.HCl pH 7.5, 150 mM NaCl, 0.5% NP40 supplemented with protease and phosphatase inhibitors (Calbiochem, San Diego, Calif.) and clarified by centrifugation. 50 μg of each sample was fractionated by SDS-PAGE, transferred to nitrocellulose membranes and probed with antibodies against the following proteins: phospho-MAPK, Total MAPK, phospho-p70S6-Kinase, (Cell Signaling Technology, MA). E-cadherin (BD Biosciences, CA) was used as a loading control. Blots were developed using Supersignal West Femto (Pierce, Rockland, Ill.). Images were captured using a Fluor Chem 8900 imager (Alpha Innotech, San Leandro, Calif.).

ELISA. Equal numbers of cells were washed three times and allowed to condition medium for 90 minutes. The amount of AREG and TGFα accumulating in the medium was determined by using specific ELISA assays (R & D Systems, Minneapolis, Minn.) according to the manufacturer's instructions.

Cloning of pro-AREG and pro-TGFα. The open reading frames of these genes were amplified by PCR from T4-2 cDNA. Amplification products were cloned, sequence verified, and subcloned into the retroviral expression vector, pBM-IRES-Puro (Garton, K. J., Ferri, N., and Raines, E. W. (2002). Efficient expression of exogenous genes in primary vascular cells using IRES-based retroviral vectors. Biotechniques 32, 830, 832, 834 passim.). The primers used were: AREG: 5′-GACCTCAATGACACCTACTCTGG-3′ (SEQ ID NO: 5), 5′-GAAATATTCTTGCTGACATTTGC-3′ (SEQ ID NO: 6); TGFα 5′-ATGGTCCCCTCGGCTGGACAGCTC-3′ (SEQ ID NO: 7), 5′-TCATAGATCTTCTTCTGATATAAGCTTTTGTTCGACCACTGTTTCTGAGTGGC-3′ (SEQ ID NO: 8). The ΔTM mutants of AREG and TGFα were generated from the using the pBM-IRES-puro specific primer 5′-TGGAAAGGACCTTACACAGTCC-3′ (SEQ ID NO: 9) and either 5′-AAAAGGATCCTCATTTTGATAAACTACTGTCAATC-3′ (AREGΔTM; SEQ ID NO: 10) or 5′-AAAAGGATCCTCAGGCCTGCTTCTTCTGGCTGGC-3′ (TGFαΔΔTM; SEQ ID NO: 11) and cloned into pBM-IRES-Puro.

Proliferation assays. HMT3522 cells were seeded in 96 well plates and treated (in triplicate) as described in the figure legends. To determine relative growth, 0.1 volumes of WST cell proliferation analysis reagent (Roche, Indianapolis, Ind.) was added to the medium and its formazan metabolite was measured by absorbance at 460 nm.

Quantitation of colony size in the 3D lrECM culture was performed by image analysis. Representative images were captured using a digital camera attached to a phase contrast microscope. The high resolution digital images were then analyzed using NIH Image analysis software and the cross-sectional area of each colony was determined for many colonies. This process was hand-curated to ensure that aggregates of colonies were not measured as a single colony.

RTPCR. DNase-treated total RNA was isolated using the RNEasy kit (Qiagen, Valencia, Calif.). 5 μg of total RNA in a final volume of 40 μl was used for oligo dT primed cDNA synthesis (First Strand cDNA synthesis kit, Invitrogen, Carlsbad, Calif.). 1 μl of cDNA was added to a 60 μl PCR reaction. 15 μl aliquots were withdrawn after 25, 30 and 35 cycles and analyzed by agarose gel electrophoresis. Primers used were as follows:

| | |
|---|---|
| AREG: | |
| 5′-GACCTCAATGACACCTACTCTGG-3′, | (SEQ ID NO: 5) |
| 5′-GAAATATTCTTGCTGACATTTGC-3′; | (SEQ ID NO: 6) |
| GAPDH: | |
| 5′-CCCCTGGCCAAGGTCATCCATGAC-3′, | (SEQ ID NO: 12) |
| 5′-CATACCAGGAAATGAGCTTGACAAAG-3′; | (SEQ ID NO: 13) |
| TACE: | |
| 5′-CAGCACAGCTGCCAAGTCATT-3′, | (SEQ ID NO: 14) |
| 5′-CCAGCATCTGCTAAGTCACTTCC-3′; | (SEQ ID NO: 15) |
| TGFα: | |
| 5′-CACACTCAGTTCTGCTTCCA-3′, | (SEQ ID NO: 16) |
| 5′-TCAGACCACTGTTTCTGAGTGGC-3′. | (SEQ ID NO: 17) |

Statistical Analyses. All data analysis was performed using Graphpad Prism. Bar graphs represent mean±standard error of mean. Significance was determined using ANOVA. In scatter plots, the horizontal bar represents the median of each dataset. Significance was determined using Kruskal-Wallis test (with Dunn's Test to correct for multiple comparisons).

A database consisting of the microarray profiles of 295 human breast tumors with the associated clinical data (van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009) was obtained from Rosetta Inpharmatics. Pearson's correlation coefficient was used to determine whether statistically significant associations existed between the relative expression levels of each of the markers. For survival analysis, patients were stratified into quartiles for expression of each marker, and survival curves computed using the method of Kaplan and Meier. Statistical significance was determined using the log-rank test.

### Example 2

### Model

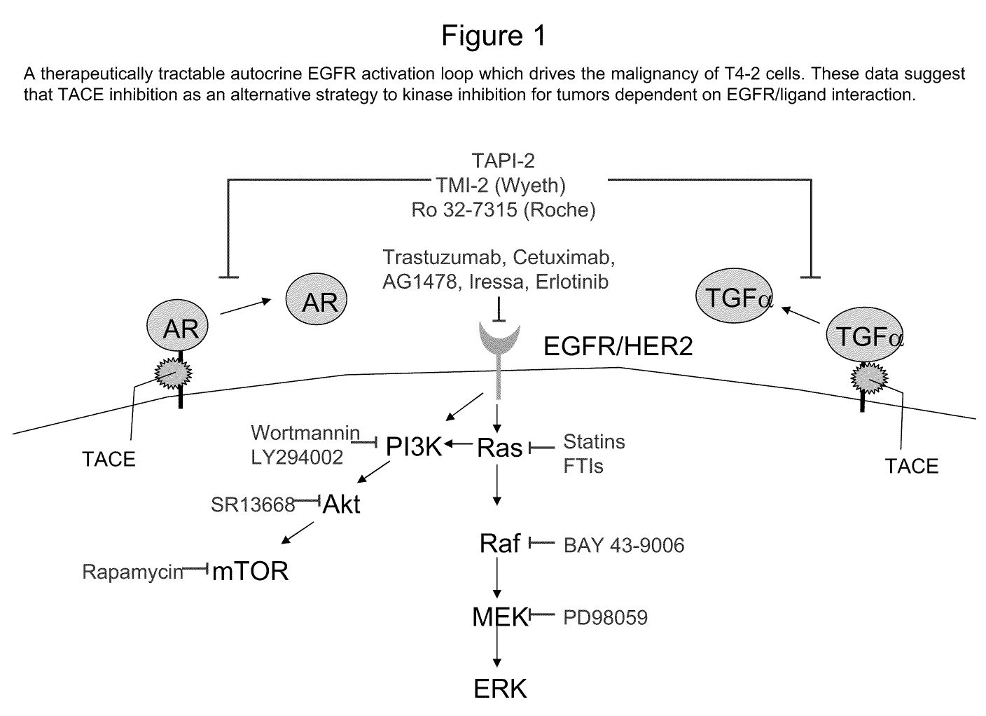

Here, we use a model (FIG. 1) to investigate the mechanisms by which non-malignant breast epithelial cells may escape dependence on exogenous EGF. A dissection of the EGFR pathway in T4-2 cells revealed that these cells lack mutations in common proto-oncogenes (H-Ras, K-Ras, N-Ras and B-Raf) but express two EGFR ligands not present in S1 cells, Amphiregulin (AREG) and TGFα. A metalloproteinase activity, TACE/ADAM 17, implicated by others in processing of these ligands (Borrell-Pages, M., Rojo, F., Albanell, J., Baselga, J., and Arribas, J. (2003). TACE is required for the activation of the EGFR by TGF-alpha in tumors. EMBO J. 22, 1114-1124; Gschwind, A., Hart, S., Fischer, O. M., and Ullrich, A. (2003). TACE cleavage of proamphiregulin regulates GPCR-induced proliferation and motility of cancer cells. EMBO J. 22, 2411-2421; Sahin, U., Weskamp, G., Kelly, K., Zhou, H. M., Higashiyama, S., Peschon, J., Hartmann, D., Saflig, P., and Blobel, C. P. (2004). Distinct roles for ADAM10 and ADAM17 in ectodomain shedding of six EGFR ligands. J Cell Biol 164, 769-779), is expressed in T4-2 cells and is necessary for AREG and TGFα function. We show that inhibition of TACE is sufficient to block EGFR signaling and to revert the malignant phenotype in T4-2 cells and that this is a direct consequence of attenuation of growth factor ectodomain shedding. We show that activation of EGFR signaling in the T4-2 cells of the HMT3522 breast cancer progression series is driven by a TACE-dependent growth factor autocrine loop not present in the non-malignant S1 cells. Inhibition of TACE attenuated the growth of T4-2 cells in 3D ECM culture and reverted their morphology to approximate that of non-malignant cells. This reversion was overcome by overexpression of soluble “pre-cleaved” mutants of either AREG or TGFα but not by pro-AREG or pro-TGFα, definitively demonstrating the importance of growth factor precursor cleavage for ErbB function. We further show that expression of TACE and TGFα is highly correlated in human breast cancer samples and is predictive of poor prognosis. Lastly, we demonstrate that ligand bioavailability, which can be modulated by TACE inhibition, is an important determinant of EGFR inhibitor efficacy.

Our data demonstrate that the utility of an EGFR inhibitor may be a function of the abundance of ligand and that low picomolar levels of these growth factors can overcome the efficacy of the inhibitor. If so, physiological levels of EGFR ligands may antagonize the action of EGFR inhibitors in vivo. Among the small proportion of patients whose tumors respond to small molecule EGFR inhibitors, there is frequent and pronounced adverse systemic reactions to the drug, including skin rash and diarrhea. Intriguingly, these systemic responses predict response to Gefitinib (Chiu, C. H., Tsai, C. M., Chen, Y. M., Chiang, S. C., Liou, J. L., and Perng, R. P. (2005). Gefitinib is active in patients with brain metastases from non-small cell lung cancer and response is related to skin toxicity. Lung Cancer 47, 129-138; Mohamed, M. K., Ramalingam, S., Lin, Y., Gooding, W., and Belani, C. P. (2005). Skin rash and good performance status predict improved survival with gefitinib in patients with advanced non-small cell lung cancer. Ann Oncol 16, 780-785; Perez-Soler, R., Chachoua, A., Hammond, L. A., Rowinsky, E. K., Huberman, M., Karp, D., Rigas, J., Clark, G. M., Santabarbara, P., and Bonomi, P. (2004). Determinants of tumor response and survival with erlotinib in patients with non-small-cell lung cancer. J Clin Oncol 22, 3238-3247). The fact that systemic toxicity predicts response is consistent with the hypothesis that circulating and locally produced EGFR ligands antagonize Gefitinib efficacy in both the tumor and in other organs: Those patients with circulating ligands above a threshold would experience neither skin rashes nor tumor regression in response to Gefitinib.

This is supported by our demonstration in 3D cultures that EGFR inhibitor efficacy is critically dependent on the bioavailability of EGFR ligands, and that ligand concentrations within the normal circulating range (Messa, C., Russo, F., Caruso, M. G., and Di Leo, A. (1998). EGF, TGF-alpha, and EGF-R in human colorectal adenocarcinoma. Acta Oncol 37, 285-289; Reeka, N., Berg, F. D., and Brucker, C. (1998). Presence of transforming growth factor alpha and epidermal growth factor in human ovarian tissue and follicular fluid. Hum Reprod 13, 2199-2205; Sotnikova, N.Y., Antsiferova, Y. S., and Shokhina, M. N. (2001). Local Epidermal Growth Factor Production in Women with Endometriosis. Russ J Immunol 6, 55-60) confer resistance to the inhibitor. Interestingly, Amphiregulin overexpression predicts non-responsiveness to Gefitinib in ERRB2-positive non-small cell lung cancer tumors (Kakiuchi, S., Daigo, Y., Ishikawa, N., Furukawa, C., Tsunoda, T., Yano, S., Nakagawa, K., Tsuruo, T., Kohno, N., Fukuoka, M., et al. (2004). Prediction of sensitivity of advanced non-small cell lung cancers to gefitinib (Iressa, ZD1839). Hum Mol Genet, 2004 Dec. 15; 13(24):3029-43. Epub 2004 Oct. 20). Undoubtedly in some cases Gefitinib resistance is acquired in a cell-autonomous fashion by tumor cells, by loss of PTEN (She, Q. B., Solit, D., Basso, A., and Moasser, M. M. (2003). Resistance to gefitinib in PTEN-null HER-overexpressing tumor cells can be overcome through restoration of PTEN function or pharmacologic modulation of constitutive phosphatidylinositol 3′-kinase/Akt pathway signaling. Clin Cancer Res 9, 4340-4346), by acquisition of mutation in EGFR (Pao, W., Miller, V. A., Politi, K. A., Riely, G. J., Somwar, R., Zakowski, M. F., Kris, M. G., and Varmus, H. (2005). Acquired Resistance of Lung Adenocarcinomas to Gefitinib or Erlotinib Is Associated with a Second Mutation in the EGFR Kinase Domain. PLoS Med 2, e73), or perhaps by switching dependence to other ErbB family members. Thus, systemic resistance to EGFR inhibitors, resulting from an excess of circulating EGFR ligands, may be an important predictive determinant for anilinoquinazoline efficacy and that reduction of the levels of circulating growth factors by inhibiting growth factor shedding may improve the efficacy of these compounds.

The demonstration of an absolute requirement for an ADAM-like proteolytic activity for proliferation in a physiologically relevant model of human breast cancer progression suggests another avenue to be explored therapeutically. MMPs and ADAMs have been studied intensively and many small molecule inhibitors have been characterized in both cell culture and animal models, primarily to inhibit MMP-dependent tumor cell invasion. Despite the relative success of pre-clinical studies, the results of many clinical trials of MMP inhibitors in cancer have been disappointing, perhaps due to what retrospectively appears to be flawed design of the Phase III studies (Coussens, L. M., Fingleton, B., and Matrisian, L. M. (2002). Matrix metalloproteinase inhibitors and cancer: trials and tribulations. Science 295, 2387-2392). It has become clear that metalloproteinases play more complex and diverse roles in tumor progression (Egeblad, M., and Werb, Z. (2002). New functions for the matrix metalloproteinases in cancer progression. Nat Rev Cancer 2, 161-174) than was appreciated during the design of these earlier clinical studies.

Our data delineate a mechanism by which breast epithelial cells may escape dependence on extrinsic proliferative signals, a transition necessary in the evolution of all cancers. The essential role of TACE in this phenotype, and the demonstration that inhibition of this protease blocks EGFR signaling and reverts the malignant phenotype suggests that interruption of such an autocrine loop might prove an effective therapy for tumors dependent on EGFR ligand expression, alone or in combination with existing EGFR inhibitors.

### Example 3

### Amphiregulin and TGFα are Upregulated in T4-2 Cells

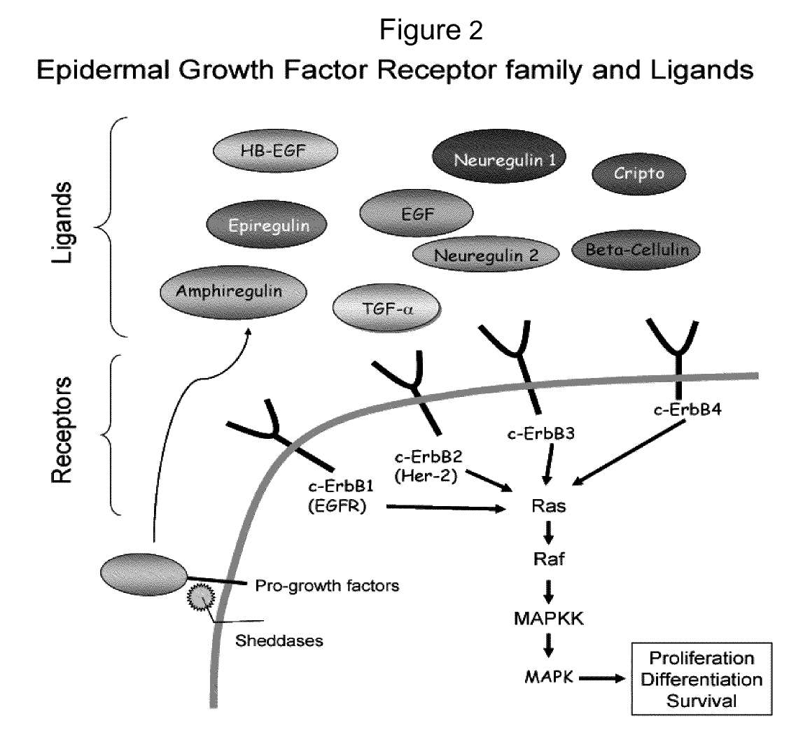

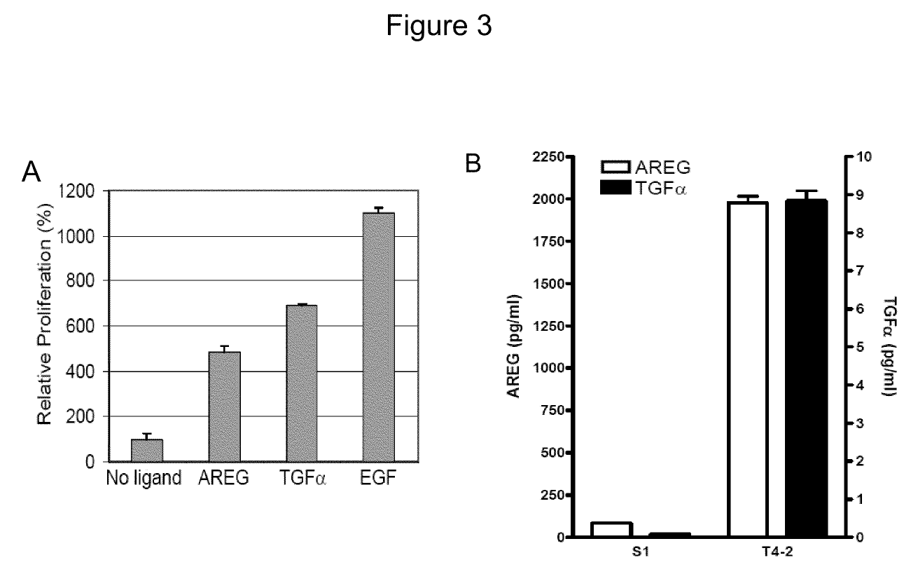

S1 non-malignant human breast epithelial cells require exogenous EGF for proliferation, while their malignant derivatives, T4-2, have acquired self-sufficiency for this signal. The sensitivity of T4-2 cells to inhibition of EGFR (Wang et al., 1998) implies that EGFR and the downstream components of the pathway are not mutationally activated. Using direct sequencing, we showed that these cells have not sustained activating mutations in H-Ras, K-Ras, N-Ras or B-Raf (data not shown). Thus, we hypothesized that T4-2 cells escaped dependence on exogenous EGF by transcriptionally upregulating one or more ErbB ligands. Conditioned medium from T4-2 cells elicited a rapid activation of MAPK in S1 cells, which was comparable to that induced by exogenously added EGF. While ligands of a number of receptor tyrosine kinases will activate MAPK (FIG. 2), the observed activation was suppressed by pre-incubation of S1 cells with the EGFR inhibitor, Iressa. Thus T4-2 cells produce one or more soluble EGFR ligands. We tested expression of Amphiregulin, Betacellulin, Cripto, EGF, Epiregulin, HB-EGF, NRG1, NRG2 and TGFα by RT-PCR. Amphiregulin and TGFα were expressed at high levels in T4-2 cells as compared to S1 cells as shown by strong bands for amphiregulin and TGFα in RT-PCR analysis. Experiments using concentrations of recombinant AREG or TGFα equimolar to that of EGF (860 μM) show that these ligands can substitute for EGF to promote proliferation of the non-malignant cells as shown by 3D culture images. FIG. 3A shows graphical results of the percentage of relative proliferation from the experiment. Using ELISA, we confirmed the presence of Amphiregulin and TGFα in the conditioned medium of T4-2 cells. (FIG. 3B).

By the time an incipient cancer cell has become malignant, it is characterized by multiple genomic mutations, chromosomal amplifications and deletions, and aneuploidy (Albertson, D. G., Collins, C., McCormick, F., and Gray, J. W. (2003). Chromosome aberrations in solid tumors. Nat Genet. 34, 369-376; Rajagopalan, H., Nowak, M. A., Vogelstein, B., and Lengauer, C. (2003). The significance of unstable chromosomes in colorectal cancer. Nat Rev Cancer 3, 695-701). In such a chaotic background, it is often difficult to distinguish between causative changes, correlative changes and changes of little consequence. If the changes we have observed in expression of these genes in malignant T4-2 cells are indeed important, one might expect that some of them would be detected at earlier stages of progression. On the continuum between S1 and T4-2 cells, a subline was established which, like S1 cells, is non-malignant but which grows independently of EGF and has lost the ability to form growth-arrested polarized acinar structures in 3D lrECM culture. This subline, S2 cells, was derived from S1 cells by EGF withdrawal after 118 passages (Briand et al., 1996). Analysis of the expression of AREG and TGFα in S2 cells by RT-PCR demonstrated that both of these factors were upregulated during the transition from S1 to S2, the earliest stage of progression toward malignancy in this series.

### Example 4

### A Metalloproteinase Activity is Critically Required for Mobilization of Growth Factors

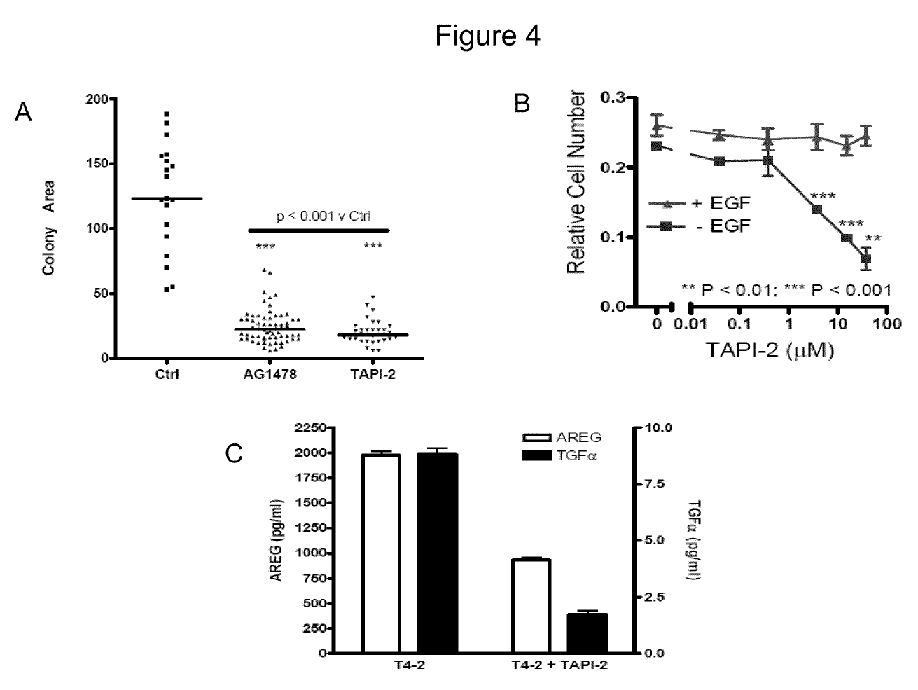

Several growth factors, including AREG and TGFα, are synthesized as transmembrane precursors and members of the ADAM family of transmembrane proteases have been implicated in the processing of these ligands (Borrell-Pages et al., 2003; Gschwind et al., 2003; Sahin et al., 2004). Culture of T4-2 cells in 3D extracellular matrix results in the formation of disorganized, apolar, continuously proliferating colonies, a phenotype we have shown to be highly correlated with, and reflective of, cancer cell malignancy (Petersen et al., 1992; Wang, F., Hansen, R. K., Radisky, D., Yoneda, T., Barcellos-Hoff, M. H., Petersen, O. W., Turley, E. A., and Bissell, M. J. (2002). Phenotypic reversion or death of cancer cells by altering signaling pathways in three-dimensional contexts. J Natl Cancer Inst 94, 1494-1503). Incubation with TAPI-2, a broad-spectrum inhibitor of MMPs and ADAMs, resulted in a reversion of the malignant phenotype similar to that elicited using the EGFR inhibitor AG1478, suggesting that a metalloproteinase activity is required for the proliferative phenotype of T4-2 cells. This treatment also resulted in the restoration of epithelial polarity. Vehicle-treated cells remained disorganized while TAPI-2 treated cells assumed a polar organization similar to that of a breast acinus, which was indicated by basal localization of α6-integrin. The colonies formed by AG1478 or TAPI-2 treated T4-2 cells were similar in size to non-malignant mammary acini and were significantly smaller than those formed by cells treated with vehicle alone (FIG. 4A).

T4-2 cells exhibit a basal level of activity of signaling kinases downstream of the EGFR which is consistent with a response to the ongoing production of an EGFR ligand by these cells. The basal activities were significantly suppressed by addition of TAPI-2 but the cells remained competent to respond to addition of exogenous EGF. Furthermore, TAPI-2 caused a dose-dependent decrease in proliferation of T4-2 cells in 2D cultures, which was overcome by addition of exogenous EGF (FIG. 4B). This compound was not cytotoxic at the concentration used, nor did it interfere with the ability of S1 cells to execute normal acinar morphogenesis in the presence of soluble EGF (data not shown). Thus, the proliferative block and concomitant reversion resulting from metalloproteinase inhibition appears to result, at least in part, from a defect in growth factor mobilization, and this again was confirmed by ELISA analysis (FIG. 4C). Thus, either an MMP or ADAM family member plays a crucial role in the regulation of EGFR signaling in this transition to growth factor autonomy.

### Example 5

### TAPI-2 Sensitizes T4-2 Cells to EGFR Inhibition

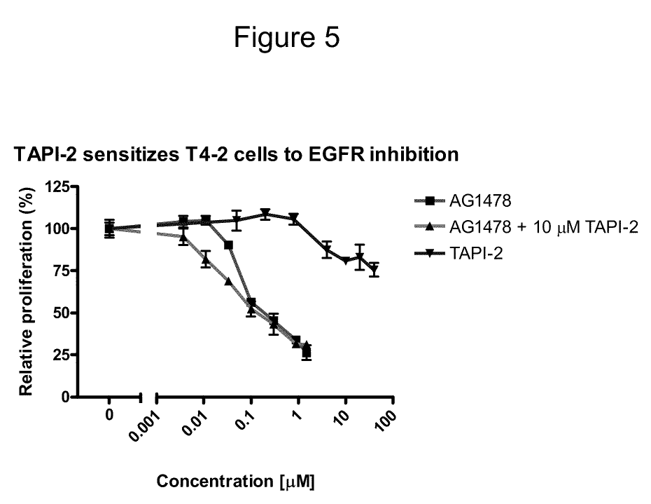

Based on relative proliferation of T4-2 cells, it was shown that TAPI-2, a TACE inhibitor, sensitizes T4-2 cells to the effect of the EGFR inhibitor, AG1478. (FIG. 5) In this experiment, T4-2 cells were allowed to adhere to 48 well plates. Drugs, AG 1478 and TAPI-2, were added at various concentrations of 0.001 μM, 0.01 μM, 0.1 μM, 1 μM, 10 μM and 100 μM. Conditions were analyzed in triplicate. For comparison, all data were normalized to the 100%. The shift of the AG 1478 and 10 uM TAPI-2 treated cell curve to the left of the curve of the cells treated only with AG 1478 demonstrates that addition of the TACE inhibitor increases the sensitivity of the cells to the EGFR inhibitor at those doses.

FIG. 5 shows that we have achieved a four-fold sensitization to small molecule EGFR inhibitors by using modest amounts of TACE inhibitors—in this case 10 μM TAPI-2. The lowest curve (▴; AG 1478 and 10 μM TAPI-2) of the graph also shows that lower dosages of an EGFR inhibitor, such as AG1478, would be required when administered with a TACE inhibitor in order to achieve the same effect with EGFR inhibitor alone. Sensitivity of the cell to AG 1478 is enhanced as shown by the reduction in relative proliferation from over 100% to about 75% when only 0.01 M AG 1478 is used versus no reduction in proliferation when the EGFR inhibitor is used alone at the same concentration. In other words, the co-application of the EGFR inhibitor with the TACE inhibitor resulted in about 25% greater effectiveness of the EGFR inhibitor, in modulation of proliferation and reduction of tumor size and growth rate, relative to the sensitivity of the cell in absence of the TACE inhibitor.

These data suggest that by modulating the bioavailability of EGFR ligands using TACE inhibition, we can effectively sensitize cells to much lower doses of small molecule EGFR inhibitors. Clinically, this suggests that the co-treatment strategy will provide an enhanced therapeutic index, allowing responses to EGFR inhibitors to occur which would otherwise not happen due to excessive levels of EGFR/ERRB family ligands in the tumor which diminish the sensitivity to clinically achievable doses of EGFR inhibitors.

### Example 6

### TACE/ADAM17 Cleaves Both AREG and TGFα in Cultured Mammary Epithelial Cells

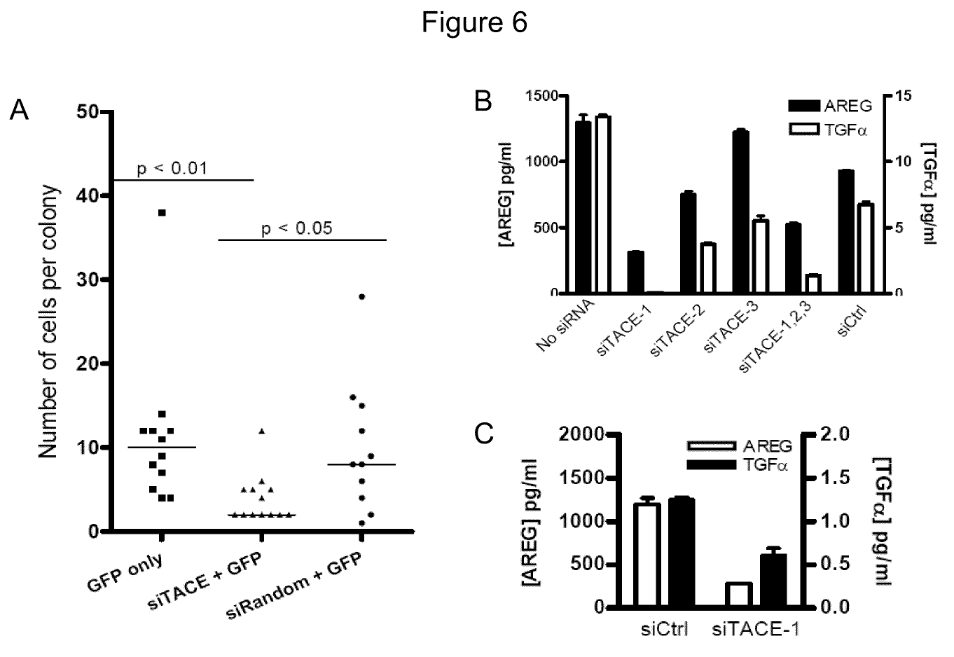

Several lines of genetic and biochemical evidence suggest that TACE/ADAM17 is a key regulator of cleavage of both AREG and TGFα (Borrell-Pages et al., 2003; Gschwind et al., 2003; Sahin et al., 2004). TACE is expressed in both S1 and T4-2 cells as shown by RT-PCR. To test whether TACE could cleave endogenously produced growth factors in mammary epithelial cells, we cloned and overexpressed the transmembrane precursors of Amphiregulin and TGFα in S1 cells. Acute stimulation of these cells with recombinant TACE was sufficient to mobilize the growth factors to activate receptor tyrosine kinase signaling, a response not elicited in the vector control cells. Introduction of siRNAs against TACE significantly suppressed T4-2 cell proliferation compared to both GFP-transfected and random siRNA-transfected controls (FIG. 6A). Thus it appears that TACE, and not another TAPI-2-sensitive protease, is the primary growth factor sheddase in T4-2 cells.

Additionally, to test whether TACE is the key sheddase for these endogenously produced growth factors in mammary epithelial cells, we used siRNA to knock down expression of TACE and measured growth factor shedding from the transfected cells (FIG. 6B). Three siRNAs against TACE were used, which suppressed TACE expression with varying degrees of efficacy. The most effective siRNA (siTACE-1) resulted in a dramatic decrease in the shedding of both ligands, while cells transfected with the less effective siRNAs retained the ability to shed ligands in proportion to the amount of TACE expressed. Introduction of the most effective siRNA against TACE had no apparent effect on morphology of cells cultured on plastic but resulted in a dramatic reversion of the malignant phenotype of T4-2 cells in 3D lrECM (laminin-rich extracellular matrix) culture compared to the random siRNA-transfected control. The shedding of both EGFR ligands was significantly reduced in these cultures (FIG. 6C). Thus it appears that TACE, and not another TAPI-2-sensitive protease, is the primary growth factor sheddase in the T4-2 breast cancer cell line.

### Example 7

### AREG and TGFα are the Key Substrates of TACE in T4-2 Cells

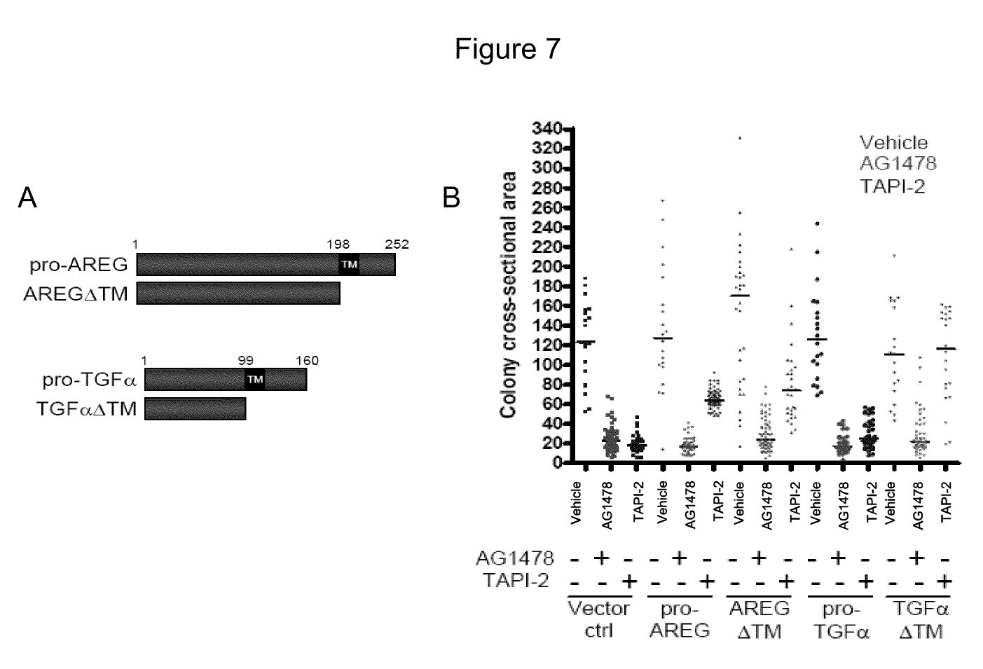

In addition to shedding growth factors, TACE has been implicated in the shedding of several cell surface molecules, inhibition of which might also contribute to the observed reversion of the T4-2 cell phenotype. Characterized substrates of TACE include TNFα (Black, R. A., Rauch, C. T., Kozlosky, C. J., Peschon, J. J., Slack, J. L., Wolfson, M. F., Castner, B. J., Stocking, K. L., Reddy, P., Srinivasan, S., et al. (1997). A metalloproteinase disintegrin that releases tumour-necrosis factor-alpha from cells. Nature 385, 729-733; Moss, M. L., Jin, S. L., Milla, M. E., Bickett, D. M., Burkhart, W., Carter, H. L., Chen, W. J., Clay, W. C., Didsbury, J. R., Hassler, D., et al. (1997). Cloning of a disintegrin metalloproteinase that processes precursor tumour-necrosis factor-alpha. Nature 385, 733-736), L-Selectin and TNFRII (Peschon, J. J., Slack, J. L., Reddy, P., Stocking, K. L., Sunnarborg, S. W., Lee, D. C., Russell, W. E., Castner, B. J., Johnson, R. S., Fitzner, J. N., et al. (1998). An essential role for ectodomain shedding in mammalian development. Science 282, 1281-1284), β-APP (Buxbaum, J. D., Liu, K. N., Luo, Y., Slack, J. L., Stocking, K. L., Peschon, J. J., Johnson, R. S., Castner, B. J., Cerretti, D. P., and Black, R. A. (1998). Evidence that tumor necrosis factor alpha converting enzyme is involved in regulated alpha-secretase cleavage of the Alzheimer amyloid protein precursor. J Biol Chem 273, 27765-27767), collagen XVII (Franzke, C. W., Tasanen, K., Schacke, H., Zhou, Z., Tryggvason, K., Mauch, C., Zigrino, P., Sunnarborg, S., Lee, D.C., Fahrenholz, F., and Bruckner-Tuderman, L. (2002). Transmembrane collagen XVII, an epithelial adhesion protein, is shed from the cell surface by ADAMs. EMBO J. 21, 5026-5035), growth hormone receptor (Zhang, Y., Jiang, J., Black, R. A., Baumann, G., and Frank, S. J. (2000). Tumor necrosis factor-alpha converting enzyme (TACE) is a growth hormone binding protein (GHBP) sheddase: the metalloprotease TACE/ADAM-17 is critical for (PMA-induced) GH receptor proteolysis and GHBP generation. Endocrinology 141, 4342-4348), TrkA (Diaz-Rodriguez, E., Montero, J. C., Esparis-Ogando, A., Yuste, L., and Pandiella, A. (2002). Extracellular signal-regulated kinase phosphorylates tumor necrosis factor alpha-converting enzyme at threonine 735: a potential role in regulated shedding. Mol Biol Cell 13, 2031-2044), ErbB4 (R10, C., Buxbaum, J. D., Peschon, J. J., and Corfas, G. (2000). Tumor necrosis factor-alpha-converting enzyme is required for cleavage of erbB4/HER4. J Biol Chem 275, 10379-10387) and GPIbα (Bergmeier, W., Piffath, C. L., Cheng, G., Dole, V. S., Zhang, Y., von Andrian, U. H., and Wagner, D. D. (2004). Tumor necrosis factor-alpha-converting enzyme (ADAM 17) mediates GPIbalpha shedding from platelets in vitro and in vivo. Circ Res 95, 677-683). To test whether modulation of growth factor cleavage is the key role of TACE here, and whether their overexpression might lead to a genetic rescue of the TAPI-2-imposed reversion, we generated soluble secreted mutants of both AREG and TGFα lacking both the transmembrane and cytosolic domains (FIG. 7A). Whereas each stably vector-transfected T4-2 cell line was susceptible to reversion by EGFR inhibition, those cells which produced soluble growth factors were completely resistant to TAPI-2 by criteria of colony size and morphology (FIGS. 7B). Like the parental cells, they continued to proliferate and formed disorganized, non-polarized colonies. Thus, despite the number of TACE substrates expressed by these cells, it is the suppression of growth factor mobilization which results in the reversion of the malignant phenotype.

### Example 8

### EGFR Ligand Bioavailability Antagonizes Inhibitor Efficacy

Like AG1478, Gefitinib (Iressa, ZD1839) and Erlotinib (Tarceva, OSI-774) are reversible anilinoquinazoline-derivatives. In two large trials of non-small cell lung cancer (NSCLC) patients, Chemotherapy with Gefitinib performed no better than chemotherapy alone in terms of survival (Giaccone, G., Herbst, R. S., Manegold, C., Scagliotti, G., Rosell, R., Miller, V., Natale, R. B., Schiller, J. H., Von Pawel, J., Pluzanska, A., et al. (2004). Gefitinib in combination with gemcitabine and cisplatin in advanced non-small-cell lung cancer: a phase III trial—INTACT 1. J Clin Oncol 22, 777-784; Herbst, R. S., Giaccone, G., Schiller, J. H., Natale, R. B., Miller, V., Manegold, C., Scagliotti, G., Rosell, R., Oliff, I., Reeves, J. A., et al. (2004). Gefitinib in combination with paclitaxel and carboplatin in advanced non-small-cell lung cancer: a phase III trial—INTACT 2. J Clin Oncol 22, 785-794). Although Erlotinib did provide a statistically significant survival benefit in patients with advanced pancreatic adenocarcinoma, it is important to note that the median extension in progression-free survival was a mere six days, while the median increase in overall survival was 14 days (Moore, M. J. et al. Erlotinib improves survival when added to gemcitabine in patients with advanced pancreatic cancer. A phase III trial of the National Cancer Institute of Canada Clinical Trials Group [NCIC-CTG]. ASCO Gastrointestinal Cancers Symposium (2005) Abstract 77). In a recent trial using Erlotinib as a single agent in NSCLC, median progression-free survival time was increased by almost two weeks in the treated population, while median overall survival was increased by two months (Shepherd, F. A., Rodrigues Pereira, J., Ciuleanu, T., Tan, E. H., Hirsh, V., Thongprasert, S., Campos, D., Maoleekoonpiroj, S., Smylie, M., Martins, R., et al. (2005). Erlotinib in previously treated non-small-cell lung cancer. N Engl J Med 353, 123-132).

Although T4-2 cells expressing soluble AREG or TGFα still formed small rounded colonies in the presence of the EGFR inhibitor, these colonies were consistently larger than those formed by cells infected with either empty vector or with the membrane-tethered pro-forms of AREG and TGFα. The difference was statistically significant (median cross-sectional area of AG1478-treated proAREG v AREGΔTM colonies, P<0.0001; proTGFα v TGFαΔTM, P<0.05). The malignant phenotype of T4-2 cells (typically cultured in the absence of EGF) may be reverted using AG1478. Increasing the amount of soluble EGF allowed the T4-2 cells to escape from the AG1478-imposed reversion. Similar observations were made with both recombinant Amphiregulin and TGFα (data not shown). Thus it seems that there is a transition point of ligand concentration above which EGFR inhibitors lack efficacy. In the 3D culture assay, this transition occurred between 0.1 and 1 ng/ml. Reported levels of EGF and TGFα (both circulating and in tumor and tissue homogenates) are within or exceed this range (Messa et al., 1998; Reeka et al., 1998; Sotnikova et al., 2001).

### Example 9

### EGFR Ligand Concentrations Affects Response of Cells to EGFR Inhibitors

It was shown that increasing amounts of the EGFR ligand, EGF, attenuates T4-2 cells response to 80 nM of AG 1478. Cells were cultured for four days in 80 nM AG1478 and various amounts of EGF. Above a threshold EGF concentration between 0.1-1 ng/ml (8.6-86 μM), the EGFR inhibitor can no longer revert the malignant phenotype. Thus, reversion of T4-2 cells by EGFR inhibition is overcome by the presence of picomolar amounts of EGFR ligand.

It was also shown that by adding TGFα, it reduced the sensitivity of T4-2 cells to Gefitinib (Iressa). Control cells were cultured in the absence of TGFα and cells were cultured in the presence of TGFα. Both the control cells and cells grown with TGFα were also cultured with various amounts of Gefitinib at 0 nM, 0.3 nM and 3 nM. Cell proliferation for cells cultured in TGFα was far more than the control cells. The addition of TGFα reduced the effectiveness of the Gefitinib.

### Example 10

### TACE Inhibition Reduces EGFR Ligand Shedding in Several Breast Cancer Cell Lines

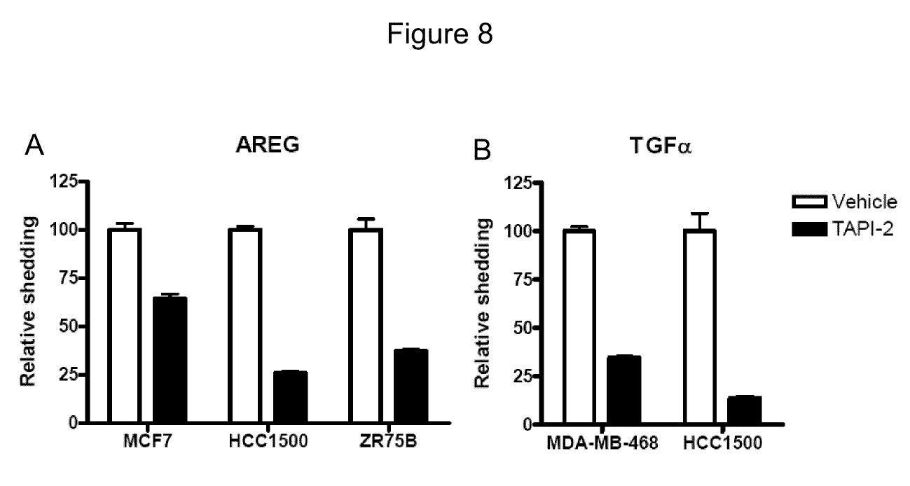

To test whether our observations were generalizable, we screened several additional breast cancer cell lines to identify those which secrete either AREG or TGFα. AREG was secreted by MCF-7, HCC1500 and ZR75B, while TGFα was secreted by HCC1500 and MDA-MB-468. In each case, 20 μM TAPI-2 significantly reduced ligand shedding from these cells (FIG. 8). Transfection of MCF-7 cells with the siRNA against TACE led to a reduction in AREG shedding by 90%, indicating that, as with T4-2 cells, TACE is the key sheddase in this cell line (data not shown). These data indicate that TACE-dependent growth factor shedding is common, at least in established breast cancer cell lines, and that it is amenable to therapeutic intervention.

### Example 11

### TACE and TGFα Predict Poor Prognosis in Human Breast Cancer Patients

Having thus established that TACE-dependent growth factor shedding plays a role that is both critically important and therapeutically tractable in this model of breast cancer progression, we sought to determine the extent to which these factors play a role in human breast cancer. We interrogated a comprehensive microarray dataset of 295 primary human breast tumors which was prepared by Marc van de Vijver and colleagues, who used it to identify gene expression signatures predictive of outcome. The detailed clinical characteristics of these tumors have been reported (van de Vijver et al., 2002). Briefly, all were either stage I or II and less than 5 cm diameter at excision, and derived from 295 consecutively treated patients less than 53 years old. Approximately three quarters of the tumors were ERα positive, and half were associated with positive lymph nodes. The median time for which follow-up information is available is 6.7 years (range 0.05-18.3 years).

Our analysis of this publicly available dataset revealed a statistically significant positive correlation between expression levels of TGFα and TACE (Table 1, P<0.001). EGFR expression also tended to correlate with both TGFα and TACE, although not quite reaching the level of statistical significance (P=0.053 and P=0.061, respectively). Interestingly, Amphiregulin expression in this patient population was inversely correlated with expression of EGFR and TGFα (P<0.05 and P<0.001, respectively). Amphiregulin and TACE levels tended to be anti-correlated, although not quite reaching statistical significance (P=0.053). While these three markers were co-expressed in our progression model, these data suggest that TACE and TGFα may be the more important protease/growth factor pair for EGFR activation in human breast tumors. Tumors positive for TGFα, ADAM17 and EGFR tended to be ERα negative (P<0.0001, P<0.005, P<0.0001 respectively). Conversely, ERα positive tumors tended to have higher levels of Amphiregulin (P<0.0001).

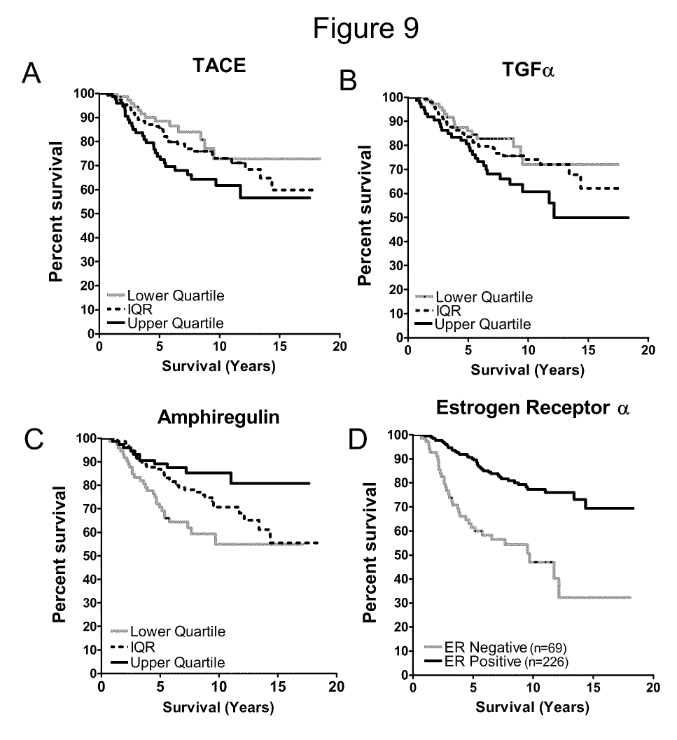

To analyze the contribution of AREG, TGFα, and TACE expression to survival, tumors were divided in quartiles by expression level of each marker and survival curves were computed for the upper and lower quartiles (74 samples each) and the interquartile range (147 samples). High levels of TACE expression were associated with a poor survival (FIG. 9A, P<0.05, high v. low expression). Tumors with the highest levels of TGFα expression also tended to have a poorer outcome, although statistically this was borderline (FIG. 9B, P<0.06). Tumors which express high levels of Amphiregulin had a significantly better outcome than tumors expressing lower levels (FIG. 9C, P<0.001) as expected from the high correlation between Amphiregulin and ERα expression levels, positivity for the latter being a strong predictor of survival (FIG. 9D, P<0.001).

TABLE 1
Pearson's correlation analysis of markers in 295 primary human breast tumors

| | AREG | | ERα | | TGFα | | ADAM17 | | EGFR | |
|---|---|---|---|---|---|---|---|---|---|---|
| | r | p | r | p | r | p | r | p | R | p |
| ERα | 0.4177 | <0.0001 | | | | | | | | |
| TGFα | −0.2155 | 0.0002 | −0.3851 | <0.0001 | | | | | | |
| ADAM17 | −0.1126 | 0.0534 | −0.1774 | 0.0022 | 0.1917 | 0.0009 | | | | |
| EGFR | −0.1416 | 0.0150 | −0.2755 | <0.0001 | 0.1127 | 0.0532 | 0.1093 | 0.0609 | | |
| ERBB2 | 0.1897 | 0.0011 | −0.0943 | 0.1062 | −0.1027 | 0.0783 | −0.0340 | 0.5609 | −0.0557 | 0.3407 |

These data illuminate the necessary steps, at a molecular level, by which tumor cells may become independent of extrinsic proliferative signals and suggest that ADAM family members may prove important additional therapeutic targets in EGFR-dependent malignancies of the breast and other tissues.

Any patents, patent publications, publications, or GenBank Accession numbers cited in this specification are indicative of levels of those skilled in the art to which the patent pertains and are hereby incorporated by reference to the same extent as if each was specifically and individually incorporated by reference.

What is claimed is:

1. A method for modulating proliferation of tumor cells, wherein the cells express TNF-α Converting enzyme (TACE), the method comprising the steps of:
(a) contacting a tumor cell with a TACE inhibitor, and
(b) contacting the cell with a compound that inhibits epidermal growth factor receptor (EGFR) tyrosine kinase,
whereby the TACE inhibitor enhances the sensitivity of the cell to the compound of step (b).

2. The method of claim 1 wherein the cell is in a mammal.

3. The method of claim 2 wherein the mammal is a human.

4. The method of claim 1 wherein the TACE inhibitor is selected from the group consisting of antisense nucleic acid molecules, anti-TACE antibodies, siRNA oligonucleotides, soluble recombinant TACE protein fragments, small molecules, peptides and peptide mimetics.

5. The method of claim 1 wherein the TACE inhibitor is a small molecule.

6. The method of claim 5 wherein the small molecule is selected from the group consisting of (E)-2(R)-[1(S)-(Hydroxycarbamoyl)-4-phenyl-3-butenyl]-2′-isobutyl-2′-(methanesulfonyl)-4-methylvalerohydrazide (Ro 32-7315), (2R,3S)-2-([[4-(2-butynyloxy)phenyl]sulfonyl]amino)-N,3-dihydroxybutanamide (TMI-2), BMS-561392 (DPC-333), N—(R)-[2-(Hydroxyaminocarbonyl)methyl]-4-methylpentanoyl-L-naphthylalanyl-L-alanine Amide (TNF-α Protease Inhibitor-0), N—(R)-[2-(Hydroxyaminocarbonyl)methyl]-4-methylpentanoyl-L-naphthylalanyl-L-alanine, 2-aminoethyl Amide (TNF-α Protease Inhibitor-1), and N—(R)-[2-(Hydroxyaminocarbonyl)methyl]-4-methylpentanoyl-L-t-butyl-alanyl-L-alanine, 2-aminoethyl Amide (TNF-α Protease Inhibitor-2).

7. The method of claim 1 wherein the TACE inhibitor is a matrix metalloproteinase inhibitor.

8. The method of claim 1 wherein the TACE inhibitor is N—(R)-[2-(Hydroxyaminocarbonyl)methyl]-4-methylpentanoyl-L-t-butyl-alanyl-L-alanine, 2-aminoethyl Amide and the compound that inhibits EGFR tyrosine kinase is AG 1478.

9. The method of claim 1 further comprising the step of contacting the cell with an anilinoquinazoline compound.

10. The method of claim 1 wherein the compound that inhibits EGFR tyrosine kinase is an antitumor therapeutic.

11. The method of claim 10, wherein the antitumor therapeutic is selected from the group consisting of: Erlotinib, Gefitinib, AG1478, Canertinib (CI-1033), EKB-569, Lapatinib (GW572016), Erbitux (Cetuximab), ABX-EGF, EMD-72000, Thera CIM-h-R3, and HuMax-EGFR.

12. The method of claim 1 wherein proliferation of the tumor cells is inhibited.

13. A method of treating cancer comprising administering to a mammal in need thereof a therapeutically effective amount of a TACE inhibitor and a therapeutically effective amount of an EGFR tyrosine kinase inhibitor, whereby the TACE inhibitor enhances the sensitivity of a cell to the EGFR tyrosine kinase inhibitor.

14. The method of claim 13 wherein the EGFR tyrosine kinase inhibitor is an antitumor therapeutic.

15. The method of claim 14, wherein the antitumor therapeutic is selected from the group consisting of: Erlotinib, Gleevec, Imatinib, Gefitinib, AG1478, CEP-1347, leflunomide, Semaxanib, Imidazo[1,2-a]pyrazin-8-ylamines, Canertinib (CI-1033), EKB-569, Lapatinib (GW572016), and monoclonal antibodies that target EGFR pathway including but not limited to, Erbitux (Cetuximab), ABX-EGF, EMD-72000, Thera CIM-h-R3, HuMax-EGFR, paclitaxel, cisplatin, carboplatin, chemotherapy, and radiation treatment.

16. The method of claim 13 wherein the TACE inhibitor is a small molecule.

17. The method of claim 16 wherein the small molecule is selected from the group consisting of (E)-2(R)-[1(S)-(Hydroxycarbamoyl)-4-phenyl-3-butenyl]-2′-isobutyl-2′-(methanesulfonyl)-4-methylvalerohydrazide (Ro 32-7315), (2R,3S)-2-([[4-(2-butynyloxy)phenyl]sulfonyl]amino)-N,3-dihydroxybutanamide (TMI-2), BMS-561392 (DPC-333), N—(R)-[2-(Hydroxyaminocarbonyl)methyl]-4-methylpentanoyl-L-naphthylalanyl-L-alanine Amide (TNF-α Protease Inhibitor-0), N—(R)-[2-(Hydroxyaminocarbonyl)methyl]-4-methylpentanoyl-L-naphthylalanyl-L-alanine, 2-aminoethyl Amide (TNF-α Protease Inhibitor-1), and N—(R)-[2-(Hydroxyaminocarbonyl)methyl]-4-methylpentanoyl-L-t-butyl-alanyl-L-alanine, 2-aminoethyl Amide (TNF-α Protease Inhibitor-2).

18. The method of claim 13 further comprising the step of administering an anilinoquinazoline compound.

19. A method of identifying a TACE inhibitor that sensitizes a tumor cell to an EGFR tyrosine kinase inhibitor, the method comprising the steps of:
(a) contacting a tumor cell with a compound suspected of being a TACE inhibitor,
(b) contacting the tumor cell with an EGFR tyrosine kinase inhibitor, and
(c) determining tumor cell proliferation,
whereby the compound that enhances the sensitivity of the tumor to the EGFR tyrosine kinase inhibitor is identified as a TACE inhibitor that sensitizes a tumor cell to an EGFR tyrosine kinase inhibitor.

20. The method of claim 19 wherein the cell is in a mammal.

21. The method of claim 20 wherein the mammal is a rodent.

22. The method of claim 19 wherein the compound suspected of being a TACE inhibitor is selected from the group consisting of antisense nucleic acid molecules, anti-TACE antibodies, siRNA oligonucleotides, soluble recombinant TACE protein fragments, small molecules, peptides and peptide mimetics.

23. The method of claim 19 wherein the compound suspected of being a TACE inhibitor is a small molecule.

24. The method of claim 23 wherein the small molecule is a matrix metalloproteinase inhibitor.

25. The method of claim 19 wherein the EGFR tyrosine kinase inhibitor is AG 1478.
